SEQUENCE LISTING (35 sequences)

SEQ ID NO: 1 - protein, 355 aa, Homo sapiens
  wingless-type MMTV integration site family, member 16, isoform 2 (Wnt16a)
MQLTTCLRETLFTGASQKTSLWWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

SEQ ID NO: 2 - protein, 365 aa, Homo sapiens
  wingless-type MMTV integration site family, member 16, isoform 1 (Wnt16b)
MDRAALLGLARLCALWAALLVLFPYGAQGNWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

SEQ ID NO: 3 - protein, 22 aa, Artificial Sequence
  Description of Artificial Sequencehuman Wnt16b antigenic peptide
AALLGLARLCALWAALLVLFPY

SEQ ID NO: 4 - protein, 14 aa, Artificial Sequence
  Description of Artificial Sequencehuman Wnt16b antigenic peptide
QKELCKRKPYLLPS

SEQ ID NO: 5 - protein, 9 aa, Artificial Sequence
  Description of Artificial Sequencehuman Wnt16b antigenic peptide
RLGIQECGS

SEQ ID NO: 6 - protein, 10 aa, Artificial Sequence
  Description of Artificial Sequencehuman Wnt16b antigenic peptide
GASPLFGYEL

SEQ ID NO: 7 - protein, 20 aa, Artificial Sequence
  Description of Artificial Sequencehuman Wnt16b antigenic peptide
TAFIYAVMAAGLVHSVTRSC

SEQ ID NO: 8 - protein, 7 aa, Artificial Sequence
  Description of Artificial Sequencehuman Wnt16b antigenic peptide
TECSCDT

SEQ ID NO: 9 - protein, 7 aa, Artificial Sequence
  Description of Artificial Sequencehuman Wnt16b antigenic peptide
SRKFLDF

SEQ ID NO: 10 - protein, 7 aa, Artificial Sequence
  Description of Artificial Sequencehuman Wnt16b antigenic peptide
NKVLLAM

SEQ ID NO: 11 - protein, 26 aa, Artificial Sequence
  Description of Artificial Sequencehuman Wnt16b antigenic peptide
GRQAVAKLMSVDCRCHGVSGSCAVKT

SEQ ID NO: 12 - protein, 8 aa, Artificial Sequence
  Description of Artificial Sequencehuman Wnt16b antigenic peptide
EKIGHLLK

SEQ ID NO: 13 - protein, 23 aa, Artificial Sequence
  Description of Artificial Sequencehuman Wnt16b antigenic peptide
RKIPIHKDDLLYVNKSPNYCVED

SEQ ID NO: 14 - protein, 9 aa, Artificial Sequence
  Description of Artificial Sequencehuman Wnt16b antigenic peptide
DGCNLLCCG

SEQ ID NO: 15 - protein, 26 aa, Artificial Sequence
  Description of Artificial Sequencehuman Wnt16b antigenic peptide
YNTHVVRHVERCECKFIWCCYVRCRR

SEQ ID NO: 16 - protein, 19 aa, Artificial Sequence
  Description of Artificial Sequencehuman Wnt16b antigenic peptide
LGIASFGVPEKLGCANLPL

SEQ ID NO: 17 - DNA, 20 bp, Artificial Sequence
  Description of Artificial Sequencehuman Wnt16a RT-PCR forward primer Wnt16aF
cagaaagatggaaaggcacc

SEQ ID NO: 18 - DNA, 20 bp, Artificial Sequence
  Description of Artificial Sequencehuman Wnt16b RT-PCR forward primer Wnt16bF
tgctcgtgctgttcccctac

SEQ ID NO: 19 - DNA, 20 bp, Artificial Sequence
  Description of Artificial Sequencehuman Wnt16a and Wnt16b RT-PCR reverse primer Wnt16aR and Wnt16bR
atcatgcagttccatctctc

SEQ ID NO: 20 - protein, 355 aa, Homo sapiens [sequence omitted]

SEQ ID NO: 21 - protein, 361 aa, Homo sapiens [sequence omitted]

SEQ ID NO: 22 - protein, 22 aa, Artificial Sequence
  Description of Artificial Sequencehuman Wnt16a N-terminal peptide
MQLTTCLRETLFTGASQKTSLW

SEQ ID NO: 23 - protein, 6 aa, Artificial Sequence
  Description of Artificial Sequencehuman Wnt16c N-terminal peptide
MERHPP

SEQ ID NO: 24 - protein, 32 aa, Artificial Sequence
  Description of Artificial Sequencehuman Wnt16b N-terminal peptide
MDRAALLGLARLCALWAALLVLFPYGAQGNWM

SEQ ID NO: 25 - DNA, 3132 bp, Homo sapiens
  wingless-type MMTV integration site family, member 16, transcript variant 1 (WNT16) precursor cDNA
cccgcatctcctgcacatctccacccctgcgcaggaggagatccccaggctgctctctccatctctcctacagctccctgcaaacgagggggaagctgctgagagtccctatcactgctggccttttaatgttgtatgcaaggaggaagagggcgagggataacttggtgctggacaactgacctgcggcccgaagggcctctggggagggggtgcaaaagaggagcggctgggctgggggactccatgcgggggcgatggacagggcggcgctcctgggactggcccgcttgtgcgcgctgtgggcagccctgctcgtgctgttcccctacggagcccaaggaaactggatgtggttgggcattgcctccttcggggttccagagaagctgggctgcgccaatttgccgctgaacagccgccagaaggagctgtgcaagaggaaaccgtacctgctgccgagcatccgagagggcgcccggctgggcattcaggagtgcgggagccagttcagacacgagagatggaactgcatgatcaccgccgccgccactaccgccccgatgggcgccagccccctctttggctacgagctgagcagcggcaccaaagagacagcatttatttatgctgtgatggctgcaggcctggtgcattctgtgaccaggtcatgcagtgcaggcaacatgacagagtgttcctgtgacaccaccttgcagaacggcggctcagcaagtgaaggctggcactgggggggctgctccgatgatgtccagtatggcatgtggttcagcagaaagttcctagatttccccatcggaaacaccacgggcaaagaaaacaaagtactattagcaatgaacctacataacaatgaagctggaaggcaggctgtcgccaagttgatgtcagtagactgccgctgccacggagtttccggctcctgtgctgtgaaaacatgctggaaaaccatgtcttcttttgaaaagattggccatttgttgaaggataaatatgaaaacagtatccagatatcagacaaaacaaagaggaaaatgcgcaggagagaaaaagatcagaggaaaataccaatccataaggatgatctgctctatgttaataagtctcccaactactgtgtagaagataagaaactgggaatcccagggacacaaggcagagaatgcaaccgtacatcagagggtgcagatggctgcaacctcctctgctgtggccgaggttacaacacccatgtggtcaggcacgtggagaggtgtgagtgtaagttcatctggtgctgctatgtccgttgcaggaggtgtgaaagcatgactgatgtccacacttgcaagtaaccactccatccagccttgggcaagatgcctcagcaatatacaatggcattgcaaccagagaggtgcccatccctgtgcagcgctagtaaagttgactcttgcagtggaatccctagaaccttggacctgagagtttcccttacctgatcgacatattttcctttatctgatcaacccatcaatcatgtggatttcttgggattctaatgttgaaaaggtttatattcaccttttgatgatttggggaatatatattgacatacaaggaagataatctgtttcctaagcaagaaataacaggaaagatcccttatgccaggaggcctgccatactcaggataagatccttgaatatggaacttagttacaggactcaataatggtgggtgaacattagtcatttttaaaagacacctcttatagcaataaggagacattaacatgaatctcatttattctctcagtattttaactgaagaaattatactgtttgtgtgtggatagaagatgttgaaaagttaacataagcattgggtgctgacttaccctttcatgtacttccaaagaaaggtaatcaaaaagaatcttcttaagtgatataatatccctaaaaaaatgatcattacagatgtttagtgacaaagaatcaatatgtaaaaagtataatgaatgatttagattttaagtgccttttcactgggagaatctggaaaaacctccataaggtatatagcaatctttgatctttagattcatacttttatcacagatcagtttcaactgttaaaaacccacctctgagatactggggggaggatcctgaaacatgcgggaaaaggagaggtaaacagtggaggtaaaaatataatttcatacattgtaaagaaaagcaccctttaaatgtgtaaagacagtgttttgtaaagaattttgtttaaaaagtttctattttgtaaatacagtacttaagttatatgatttatattaaaacatttattgacaaagcctaagagctaaggcagtaaaattatctcataaataatattagcttattttttttcatactattaatgctatttttttggacatcgaagagaatttaacttagcagttagttatatggatgtgtatttcttgctaaaatgacagttttatatgttatagattaaaatatgttgcaaaatatcaaaaatttgtgttatttcagcagtaagattaattgaattctcttttcacattagttatgcttaactcataaggttattataataaattatattagtaaaagtcttaactggaaaaaagaatctaaatcagaatagtgatcaatttgtggatttgatatcctggatatttattatattttatgtaatgctgcatttctatttgaatgttaagtggtctttcttgtttttaatattcatgcatgtatattcatcatattttacaaggttcctggtaaaaattacagggctctatttaaggatgtattttaatgtaaatgcttatgttttttatgaattgttaaatatttcagtattatatagaaaaaaatagatttttaaaattcagaatggacaaagagaatattcattttcttattaataagataaagaaatgtttccctgccccacagtcttcattctatttctctttaattttattcactgaggcagagaaacaatttttgaaaaagagcaaacccatggaaaatgtctcagatctaatattaaaatcaagactaagcatttaactgtgaaaaaaaaaaaaaaaaaaa

SEQ ID NO: 26 - DNA, 21 bp, Artificial Sequence
  Description of Combined DNA/RNA MoleculeWnt16a silencing siRNA targeting specifically Wnt16a
agauggaaaggcacccacctt

SEQ ID NO: 27 - DNA, 21 bp, Artificial Sequence
  Description of Combined DNA/RNA MoleculeWnt16a silencing siRNA targeting Wnt16a specifically
ggugggugccuuuccaucutt

SEQ ID NO: 28 - DNA, 21 bp, Artificial Sequence
  Description of Combined DNA/RNA MoleculeWnt16b1 silencing siRNA targeting Wnt16b specifically
uggcauugcaaccagagagtt

SEQ ID NO: 29 - DNA, 21 bp, Artificial Sequence
  Description of Combined DNA/RNA MoleculeWnt16b1 silencing siRNA targeting Wnt16b specifically
cucucugguugcaaugccatt

SEQ ID NO: 30 - DNA, 21 bp, Artificial Sequence
  Description of Combined DNA/RNA MoleculeWnt16b2 silencing siRNA targeting Wnt16b specifically
ggaaacuggaugugguuggtt

SEQ ID NO: 31 - DNA, 22 bp, Artificial Sequence
  Description of Combined DNA/RNA MoleculeWnt16b2 silencing siRNA targeting Wnt16b specifically
ccaaccacauccaguuuccdtt

SEQ ID NO: 32 - DNA, 21 bp, Artificial Sequence
  Description of Combined DNA/RNA MoleculeWnt16b3 silencing siRNA targeting Wnt16b specifically
ugcaaccguacaucagaggtt

SEQ ID NO: 33 - DNA, 21 bp, Artificial Sequence
  Description of Combined DNA/RNA MoleculeWnt16b3 silencing siRNA targeting Wnt16b specifically
ccucugauguacgguugcatt

SEQ ID NO: 34 - protein, 30 aa, Artificial Sequence
  Description of Artificial Sequencesingle chain Fv (scFv) antibody peptide linker
GGGGSGGGGSGGGGSGGGGSGGGGSGGGGS

SEQ ID NO: 35 - DNA, 46 bp, Artificial Sequence
  Description of Artificial Sequenceportion of Wnt16b first exon known to be different from Wnt16a first exon
ttgctcgtgccttgtcccctacggagcccaaggaaactggatgtga

(19) United States
(12) Patent Application Publication
(10) Pub. No.: US 2008/0267951 A1
(43) Pub. Date: Oct. 30, 2008

(54) Methods for Treating Cancer Using Agents That Inhibit Wnt16 Signaling

(75) Inventors: Liang You, San Francisco, CA (US); Biao He, South San Francisco, CA (US); Zhidong Xu, San Francisco, CA (US); David M. Jablons, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regets of the University of Califoria Office of Technology Management, UCSF, San Francisco, CA (US)

(21) Appl. No.: 11/630,085

(22) PCT Filed: Jul. 11, 2005
(86) PCT No.: PCT/US05/24759

§ 371 Date: Dec. 18, 2006

Related U.S. Application Data
(60) Provisional application No. 60/586,645, filed on Jul. 9, 2004.
(60) Provisional application No. 60/645,709, filed on Jan. 20, 2005.

(51) Int. Cl. A61K 39/395; A61K 31/7088; A61P 35/00; C12Q 1/68

(52) U.S. Cl. 424/133.1; 514/44; 424/138.1; 435/6; 424/178.1

(57) ABSTRACT

This invention relates to methods of inhibiting the growth of cells, in particular cancer cells that over express Wnt16 protein. The methods comprise contacting the cell with an agent that binds to Wnt16 mRNA or Wnt16 protein, interferes with Wnt16 signaling or inhibits binding of the Wnt16 protein to another protein, such as a Frizzled receptor.

### CROSS-REFERENCE TO RELATED APPLICATIONS

This application claims benefit of provisional application Ser. No. 60/586,564, filed Jul. 9, 2004 and provisional application Ser. No. 60/645,709, filed Jan. 20, 2005, the disclosures of which are incorporated in its entirety herein by reference.

### FIELD OF THE INVENTION

This invention relates to methods of inhibiting the growth of cancer cells that overexpress Wnt16 protein. The methods comprise contacting the cell with an agent that binds to Wnt16 mRNA or Wnt16 protein, interferes with Wnt16 signaling, or inhibits binding of the Wnt16 protein to other proteins, such as the Frizzled receptor.

### BACKGROUND OF THE INVENTION

Lung Cancer is the leading cause of cancer death in the United States and worldwide, with >170,000 newly diagnosed cases each year in the US and nearly a million cases worldwide (Minna et al. Cancer Cell. 1(1):49-52 (2002)). Despite aggressive approaches made in the therapy of lung cancer in the past decades, the 5-year survival rate for lung cancer remains under 15% (Minna et al. Cancer Cell. 1 (1):49-52 (2002)). Lung cancers are divided into two groups: non-small-cell lung cancer (NSCLC) and small-cell lung cancer (SCLC). NSCLC (75-80% of all cancers) consists of three major types: adenocarcinoma, squamous cell carcinoma, and large cell carcinoma (Minna (2002)). Lung carcinomas and squamous cell carcinomas represent 60-70% of all lung cancers. Surgery, chemotherapy, and radiation have been used with generally unsatisfactory results in advanced disease. Improvement in the efficacy of lung cancer treatment is a major public health goal.

Malignant pleural mesothelioma (MPM) is a highly aggressive and challenging cancer arising primarily from the pleural lining of the lung. Approximately 3,000 patients are diagnosed with MPM in the United States annually and the incidence of this tumor is predicted to increase dramatically over near term, peaking around 2020 (Thatcher, Lung Cancer 45 Suppl 1:S1-2 (2004)). Since MPM usually presents at an advanced stage, a curative resection is rarely possible. Radiotherapy has failed to show clinical benefit as a single treatment modality, and the administration of chemotherapy is mostly restricted to the advanced stage with limited efficiency (Kindler, Lung Cancer 45 Suppl 1:S125-7 (2004)). Alternative strategies based on pleural injections of recombinant cytokines have similarly proven unsatisfactory (Bard et al. Lung Cancer 45 Suppl 1:S129-31 (2004)). Since current interventions offer only limited benefit, and overall survival is low, there is an urgent need to develop new therapeutic agents based on a greater understanding of MPM's underlying molecular mechanisms.

Molecular pathogenesis of lung cancer and MPM includes alterations of expression and function of multiple genes, involving dominant oncogenes and recessive tumor suppressor genes, and abnormalities in cell signaling transduction pathways. A better understanding of molecular mechanisms for lung cancer and MPM pathogenesis should improve the treatment of patients with lung cancer.

The Wingless-type (Wnt) family of secreted glycoproteins is a group of signaling molecules broadly involved in developmental processes and oncogenesis (Polakis, Genes Dev. 14:1837-51 (2000); Lustig et al. J. Cancer Res. Clin. Oncol. 129:199-221 (2003)).

Nineteen human Wnt proteins have thus far been identified. Transduction of Wnt signals is triggered by the binding of Wnt ligands to two distinct families of cell-surface receptors: the frizzled (Fz) receptor family and the LDL-receptor-related protein (LRP) family (Akiyama, Cytokine Growth Factor Rev. 11:273-82 (2000)). Intracellularly, Wnt signaling activates dishevelled (Dvl) proteins, which inhibit glycogen synthase kinase-3β (GSK-3β) phosphorylation of β-catenin leading to its cytosolic stabilization. Stabilized β-catenin then enters the cell nucleus and associates with LEF/TCF transcription factors. β-catenin-Tcf/Lef induces transcription of important downstream target genes, many of which have been implicated in cancer. In the absence of Wnt signals, free cytosolic β-catenin is incorporated into a complex consisting of Axin, the adenomatous polyposis coli (APC) gene product, and glycogen synthase kinase (GSK)-3β. Conjunctional phosphorylation of Axin, APC, and β-catenin by GSK-3 designates β-catenin for the ubiquitin pathway and degradation by proteasomes (Uthoff et al., Int J Oncol 19(4):803-10 (2001); Matsuzawa et al., Mol Cell 7(5):915-26 2001)).

Disheveled (Dvl) is a positive mediator of Wnt signaling positioned downstream of the frizzled receptors and upstream of β-catenin. GSK-3 phosphorylates several proteins in the Wnt pathway and is instrumental in the downstream regulation of β-catenin. Mutations in the gene APC are an initiating event for both sporadic and hereditary colorectal tumorigenesis. APC mutants are relevant in tumorigenesis, since the aberrant protein is an integral part of the Wnt-signaling cascade. The protein product contains several functional domains acting as binding and degradation sites for β-catenin. Mutations that occur in the amino-terminal segment of β-catenin are usually involved in phosphorylation-dependent, ubiquitin-mediated degradation and, thus, stabilize β-catenin. When stabilized cytoplasmic-catenin accumulates, it translocates to the nucleus interacting with the Tcf/Lef high-mobility group of transcription factors that modulate expression of oncogenes such as c-myc.

It is known that Wnt/β-catenin signaling promotes cell survival in various cell types (Orford et al., J Cell Biol 146(4):855-68 (1999); Cox et al., Genetics 155(4):1725-40 (2000); Reya et al., Immunity 13(1):15-24 (2000); Satoh et al., Nat_{—}Genet. 24(3):245-50 (2000); Shih et al., Cancer Res 60(6):1671-6 (2000); Chen et al., J Cell Biol 152(1):87-96 (2001); Ioannidis et al., Nat_{—}Immunol 2(8):691-7 (2001)). Wnt signaling pathway is also thought to be associated with tumor development and/or progression (Bienz et al., Cell 103(2):311-20 (2000); Cox et al., Genetics 155(4):1725-40 (2000); (Polakis, Genes Dev 14(15):1837-51 (2000); You t al., J Cell Biol 157(3): 429-40 (2002)). Aberrant activation of the Wnt signaling pathway is associated with a variety of human cancers, correlating with the overexpression or amplification of c-Myc (He et al., Science 281(5382):1509-12 (1998); Miller et al., Oncogene 18(55):7860-72 (1999); Bienz et al., Cell 103(2):311-20 (2000); (Polakis, Genes Dev 14(15):1837-51 (2000); Brown, Breast Cancer Res 3(6):351-5 (2001)). In addition, c-Myc was identified as one of the transcriptional targets of the β-catenin/Tcfin colorectal cancer cells (He et al., Science 281(5382):1509-12 (1998); Miller et al., Oncogene 18(55):7860-72 (1999); You et al., J Cell Biol 157(3): 429-40 (2002)).

In addition to the Wnt ligands, a family of secreted Frizzled-related proteins (sFRPs) has been isolated. sFRPs appear to function as soluble endogenous modulators of Wnt signaling by competing with the membrane-spanning Frizzled receptors for the binding of secreted Wnt ligands (Melkonyan et al., Proc Natl Acad Sci USA 94(25): 13636-41 (1997)). sFRPs can either antagonize Wnt function by binding the protein and blocking access to its cell surface signaling receptor, or they can enhance Wnt activity by facilitating the presentation of ligand to the Frizzled receptors (Uthoff et al., Int J Oncol 19(4):803-10 (2001)). sFRPs seem to modulate apoptosis susceptibility, exerting an antagonistic effect on programmed cell death. To date, sFRPs have not yet been linked causatively to cancer. However, sFRPs are reported to be hypermethylated with a high frequency in colorectal cancer cell lines and this hypermethylation is associated with a lack of basal sFRP expression (Suzuki et al., Nat Genet. 31(2):141-9 (2002)).

Another protein called Dickkopf (Dkk) is also found to interfere with Wnt signaling and diminish accumulation of cytosolic β-catenin (Moon et al., Cell 88(6):725-8 (1997); Fedi et al., J Biol Chem 274(27):19465-72 (1999)). Dkk-1 antagonizes Wnt-induced signals by binding to a LDL-receptor-related protein 6 (LRP6) adjacent to the Frizzled receptor (Nusse, Nature 411 (6835):255-6 (2001)). Overexpression of Dkk-1 is also found to sensitize brain tumor cells to apoptosis (Shou et al., Oncogene 21(6):878-89 (2002)).

The effects of Wnt proteins on cell proliferation and tumor growth seem to depend on Wnt proteins interacting with their cognate cell surface receptors and subsequently inducing downstream signaling. With Wnt proteins being secreted ligands antibodies may be used to interfere with or inhibit Wnt binding to its cell surface receptor and thus affect downstream signaling. Several antibodies against Wnt proteins have been generated. For example, anti-Wnt1 (G-19) (sc-6280; Santa Cruz Biotechnology, Inc.) and anti-Wnt2 (H-20) (sc-5208; Santa Cruz Biotechnology, Inc.) are goat polyclonal antibodies raised against peptides mapping near the N-terminus of human Wnt1 and Wnt2 proteins, respectively. Wnt2 (V-16) is a goat polyclonal antibody raised against a peptide mapping within an internal region of Wnt2 of human origin (sc-5207; Santa Cruz Biotechnology, Inc.). Anti-Wnt1 and anti-Wnt2 antibodies are also described in WO 2004/032828.

Another area of interest regarding the Wnt proteins is the acute leukemias that are often characterized by translocations that can determine prognosis and treatment options. Recently, it was reported that translocations in acute myeloid leukemia could activate the Wnt signaling pathway (Muller-Tidow et al., Mol Cell Biol 24:2890-4 (2004)) Among these chromosomal abnormalities, the t(1;19) translocation results in the production of chimeric E2A-Pbx1 proteins that display oncogenic properties (Kamps et al., Genes Dev 5:358-68 (1991)). The molecular properties of E2a-Pbx1 have been extensively characterized and several candidate target genes have been identified. Light has been shed on Wnt16 in recent studies. Representational differential expression analysis performed in leukemia cell lines containing the fusion protein vs others lacking E2a-Pbx1 identified Wnt16 as a putative target gene (McWhirter et al., Proc Natl Acad Sci USA 96:11464-69 (1999)). Moreover, gene expression profiling of leukemic blasts showed a marked overexpression of Wnt16 in E2A-Pbx1-expressing leukemias (Ross et al., Mol Cell 12:393-400 (2003)). Nevertheless, the specific role played by Wnt16 and the Wnt-related proteins in acute leukemia has never been addressed so far.

Also recently, two isoforms of Wnt16, Wnt16a and Wnt16b, were identified (Fear et al., Biochem Biophys Res Commun 278:814-820 (2000)). These isoforms seem to be generated from different mRNA isoforms encoding Wnt16 protein isoforms differing at their 5′ termini. Significant levels of Wnt16a expression is observed in the pancreas, whereas Wnt16b is expressed more ubiquitously with highest levels in adult kidney, placenta, brain, heart and spleen (Fear et al., Biochem Biophys Res Commun 278(3):814-20 (2000)). Wnt16 expression has also been observed in peripheral lymphoid organs, such as spleen, appendix, and lymph nodes, but not in bone marrow (McWhirter et al., Proc Natl Acad Sci USA, 96(20):11464-9 (1999)). However, high levels of Wnt16 have been observed in bone marrow and cell lines derived from pre-B acute lymphoblastoid leukemia (ALL) patients and it has been suggested that the aberrant expression of Wnt16 is a key step in the development of t(1;19) pre-B ALL (McWhirter et al., Proc Natl Acad Sci USA, 96(20):11464-9 (1999)). Compared to normal B cells, higher expression of Wnt16 was also observed in B cell chronic lymphocytic leukemia (CLL; Lu et al., Proc Natl Acad Sci USA 101(9):3118-23 (2004)).

Despite recent advances in the understanding of Wnt signaling, the role of the Wnt16 pathway and in particular the respective role of Wnt16 isoforms in oncogenesis is unclear and has yet to be elucidated. Thus, the prior art fails to provide clear evidence that compounds that modulate the Wnt16 pathway could be useful for treatment of cells, in particular cancer cells, overexpressing Wnt16. The present invention addresses these and other needs.

### BRIEF SUMMARY OF THE INVENTION

The present invention is based, at least in part, on the discovery that the Wnt16 gene is activated in a large fraction of pediatric pre-B acute lymphoblastoid leukemia (ALL) through t(1:19) chromosomal translocation. The t(1:19) translocation produces a chimeric transcription factor (E2A-Pbx1) which causes aberrant activation of the Wnt16 gene, usually the Wnt16b gene. Wnt16 then acts in an autocrine fashion to perturb its normal cell functions and leads to pre-B ALL.

This invention provides a method of inhibiting the proliferation of a cell that overexpresses a Wnt16. The method comprises contacting the cell with an amount of an agent that inhibits Wnt16 signaling effective to inhibit proliferation of the cell.

In some embodiments, the cell is a cancer cell. The cancer cell is selected from the group consisting of lung, mesothelioma, melanoma, colon, brain, breast, ovarian, cervical, leukemia, lymphoma and non-small lung cancer cells.

A preferred cancer cell is a leukemia cell and in particular those comprising a t(1;19) translocation. In some embodiments, the leukemia cell is an acute lymphoblastoid cell, a pre-B-cell acute lymphoblastoid leukemia cell or a B cell chronic lymphocytic leukemia cell.

Another preferred cancer cell is a lung cancer cell. Also preferred is a breast cancer cell.

In one embodiment, the agent is a siRNA. In some embodiments, the agent is an anti-Wnt16 antibody, for example, an antibody that specifically binds to the Wnt16 protein, preferably a human Wnt16 protein.

Preferred are anti-Wnt16 antibodies that bind to a polypeptide comprising an amino acid sequence as shown in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16.

The invention further provides an anti-Wnt16 antibody that specifically binds a polypeptide consisting of an amino acid sequence corresponding to amino acid residues 1-99 of human Wnt16 (SEQ ID NO:2). Another anti-Wnt16 antibody specifically binds a polypeptide consisting of amino acid residues 1-99 of human Wnt16 (SEQ ID NO:2).

Also preferred is an anti-Wnt16 antibody that competes for binding a Wnt16 protein with a second anti-Wnt16 antibody that specifically binds to a polypeptide comprising an amino acid sequence as shown in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16.

Another preferred anti-Wnt16 antibody is an antibody that competes for binding a Wnt16 protein with a second anti-Wnt16 antibody that specifically binds to a polypeptide consisting of an amino acid sequence corresponding to amino acid residues 1-99 of human Wnt16 as shown in SEQ ID NO:2. Another anti-Wnt16 antibody competes for binding a Wnt16 protein with a second anti-Wnt16 antibody that specifically binds to a polypeptide consisting of amino acid residues 1-99 of human Wnt16 as shown in SEQ ID NO:2.

Antibodies of the invention can be polyclonal and monoclonal antibodies and can be prepared and modified in a number of ways. For example, the antibody may be recombinantly produced. Preferred is an anti-Wnt16 monoclonal antibody, such as a mouse monoclonal antibody. In some embodiments, the anti-Wnt16 antibody is a chimeric, a humanized antibody, a single chain Fv fragment or a fully human antibody. Particularly preferred is a human anti-Wnt16 Fab antibody.

Methods of the present invention can be practiced in vitro and/or in vivo.

The invention also provides therapeutic methods of treating cancer. In these embodiments, the cancer cell is in a patient and the step of contacting the cell is carried out by administering the agent to the patient. The method may further comprise administering to the patient a second therapeutic agent, such as a chemotherapeutic agent or radiation therapy.

The invention further provides a method of inducing apoptosis of a cell that overexpresses a Wnt16. This method comprises contacting the cell with an amount of an agent that inhibits Wnt16 signaling effective to induce apoptosis of the cell. In another aspect a method of inhibiting Wnt16 signaling in a cell is provided. This method comprises contacting the cell that overexpresses a Wnt16 with an amount of an anti-Wnt16 antibody or a Wnt16 siRNA effective to inhibit Wnt16 signaling.

In a preferred embodiment of the present invention, a method of treating a disease associated with Wnt16 signaling is provided. This method comprises administering to a subject in need of such treatment an amount of an agent that inhibits Wnt16 signaling effective to treat the disease. The agent can be an anti-Wnt16 antibody or aWnt16 siRNA. The disease can be a cancer, preferably a cancer selected from the group consisting of lung cancer, mesothelioma, melanoma, colon cancer, brain cancer, breast cancer, ovarian cancer, cervical cancer, leukemia, lymphoma and non-small lung cancer cells.

Further, the present invention provides a method of detecting in a biological sample from a patient a cell that overexpresses a Wnt16, the method comprising the step of detecting the level of Wnt16 expression in the biological sample. Preferred biological samples are blood, sputum, urine or stool and particularly preferred is serum.

In one aspect of this method, the step of detecting the level of Wnt16 expression is carried out by detecting the level of a Wnt16 mRNA. The Wnt16 mRNA can be either a Wnt16a, Wnt16b or Wnt16c mRNA. In another aspect, the step of detecting the level of Wnt16 expression is carried out by detecting the level of a Wnt16 protein.

The detection of the level of Wnt16 expression can be used to predict the response of a patient or individual to a therapeutic regimen. In one embodiment, the therapeutic regimen comprises administering to a patient a monoclonal anti-Wnt16 antibody.

Further, this invention provides pharmaceutical compositions comprising an anti-Wnt16 antibody or a Wnt16 siRNA and a pharmaceutically acceptable excipient, carrier and/or diluent. The antibody can be further conjugated to an effector component, such as a fluorescent label, a radioisotope or a cytotoxic chemical.

Methods, antibodies, pharmaceutical compositions and kits of the invention embrace the specifics as described herein.

### BRIEF DESCRIPTION OF THE DRAWINGS

FIG. 1 shows a sequence alignment of the amino acid sequences for Wnt16 isoforms. SEQ ID NO:1 corresponds to GenBank Accession No. NP_{—}057171; SEQ ID NO:20 corresponds to GenBank Accession No. EAL24346; SEQ ID NO:22 corresponds to GenBank Accession No. AAD3380352; and SEQ ID NO:2 corresponds to GenBank Accession Nos. NP_{—}476509, AAD49351, Q9UBV4 and EAL24347. “*” indicate identical amino acid residues in all 4 sequences shown. “” indicate sequences identical in SEQ ID NOS 1, 20 and 21 that are different in SEQ ID NO:2. Antigenic peptides described herein are underlined. Amino acid residues 1-99 of Wnt16 used to generate the human Fabs described herein are indicated in bold.

FIG. 2 shows Wnt signaling pathway gene expression profiling in leukemia cell lines. A. RNA was first extracted from leukemia cell lines. Both of them, 697 and RCH-ACV contain the t(1;19) translocation whereas CCL-119 do not and serves as a control. Only the data obtained from CCL-119 and 697 are shown here. After extraction, RNA was subjected to a reverse transcriptase reaction and cDNA probes were labeled with Biotin-16-dUTP, and hybridized with the Wnt specific arrays. Detection was done using a chemiluminescent reaction and the membranes were exposed to X-ray film. Wnt16 is surrounded by a black square. The genes that are upregulated in the t(1;19)-containing cell line are surrounded by black circles on the arrays. B. Data were then matched against the gene list of the GEArray Q series human Wnt signaling pathway array provided by the manufacturer. C. Upregulated and downregulated genes in both cell lines are detailed.

FIG. 3 shows expression of Wnt16 in leukemia cell lines. A. RT-PCR was performed for the four cell lines (697 and RCH-ACV contain the t(1;19) translocation whereas CCL-119 and NALM-6 do not) using specific primers for Wnt16a (negative results, not shown) and Wnt16b. The fragment of human Wnt16b amplified is 236 bp Actin primers were used to amplify β-actin as a control. B. Western-blot analysis was also performed. Proteins from all four cell lines were extracted and an equal amount (20-30 μg/lane) was subjected to immunoelectrophoresis and probed with a commercially available polyclonal antibody against Wnt16. Membranes were reprobed with an anti-β-actin antibody as a control. C. Confirmation by sequence analysis that the amplified PCR band in (a) corresponds strictly to Wnt16b. A portion of the first exon of Wnt16b which is known to be different from the first exon of Wnt16a is shown.

FIG. 4 shows Wnt16b inhibition by siRNA. A. Cells were transfected with Wnt16b-specific siRNA (cont: non-silencing siRNA, 16a: Wnt16a specific siRNA and 16b: Wnt16b specific siRNA) and after 3 days, RNA was extracted, quantified and RT-PCR was performed using the same primers as previously described. Actin serves as a control. The same cells were also transfected according to the same procedure. Proteins were extracted 3 days after the transfection and Western-Blot was performed with an anti-Wnt16 polyclonal antibody. B. Cells were transfected with control siRNA, Wnt16a siRNA and Wnt16b siRNA and 3 days later were subjected to an apoptotic assay by flow cytometry as described in the Examples. The mean value of apoptotic cell rate was calculated from 4 independent experiments. The bar graph shows the average of apoptotic rate and error bars are SD. Apoptosis analysis by flow cytometry is reported. X-axis (FL1-H) represents annexin V-FITC staining and Y-axis (FL-3H) represents propidium iodide (PI) staining. The upper row shows cells lines treated by control siRNA and the lower row shows the same cell lines treated with Wnt16b siRNA. C. Cell lines were transfected with control siRNA, Wnt16a siRNA and Wnt16b siRNA and 3 days after the transfection, proteins were extracted and a Western Blot analysis was performed with dvl-2, β-catenin and survivin antibodies as described in the Examples. Actin blotting was done as a control. These experiments have been performed 3 times with similar results. D. Wnt signaling specific arrays were performed as previously described. Some differentially-expressed genes after Wnt16b siRNA transfection are shown in CCL-119 and 697 cell lines. Non-silencing siRNA was used as a control (cont.).

FIG. 5 shows that Wnt16 antibody induces apoptosis through the canonical Wnt pathway. NALM-6 and RCH-ACV cells were treated with a mouse anti-Wnt16 antibody (BD Phanningen). Cells were plated in six-well plates and treated the day after as following: control antibody (white bar), Wnt16 antibody alone (light grey bar) and Wnt16 antibody plus Wnt16 cDNA (dark bar) as described in the Examples. At 4 days after the treatment, cells were collected, stained by propidium iodide and annexin V-FITC and subjected to flow cytometry. The mean value was calculated from three independent experiments. The bar graph shows the average of apoptotic rate and error bars are s.d.

FIG. 6 shows ELISA assays testing the biological activity and specificity of the human anti-Wnt16 Fabs disclosed herein. The respective Fab clones are indicated by numbers 582 through 589.

FIG. 7 shows human anti-Wnt16 Fabs binding to the surface of RCH-ACV cells as determined by flow cytometry.

FIG. 8 shows that an anti-Wnt16 antibody induces apoptosis through the canonical Wnt pathway. A. CCL-119 and RCH-ACV cells were treated with a custom-made human Wnt16 antibody (Fab clone #584; see, Examples). Cells were plated in 6 well plates and treated the day after as following: control antibody, Wnt16 antibody at 1 μg/ml and Wnt16 antibody at 5 μg/ml. Four days after the treatment, cells were collected, stained by propidium iodide and annexin V-FITC and subjected to flow cytometry. The mean value of apoptotic cell rate was calculated from 3 independent experiments. The bar graph shows the average of apoptotic rate and error bars are SD. B. Proteins were extracted 4 days after the Wnt16 antibody treatment and Western Blot analysis was performed using antibody against β-catenin, survivin and dvl-2.

FIG. 9 shows induction of apoptotic cell death after treatment with a mouse anti-Wnt16 monoclonal antibody in human lung cancer cell line H460. H460 cells were treated for three days with no antibody (untreated), control IgG antibody (Conl Ab; 5 ug/mL), or anti-Wnt16 monoclonal antibody (Wnt16 Ab; 5 ug/mL; BD Pharmingen). Apoptosis was analyzed using the flow cytometry. Treatment with the anti-Wnt16 monoclonal antibody induced apoptosis in 75.9% cells, as compared with 5.59% and 5.86% observed from cells untreated or treated with a control IgG antibody, respectively.

FIG. 10 shows induction of apoptotic cell death after treatment with a human anti-Wnt16 antibody (Fab clone #585) in human lung cancer cell line H460. H460 cells were treated for three days with no antibody (untreated), control IgG antibody (Conl Ab; 5 ug/mL), or anti-Wnt16 antibody (Wnt16 Ab; 5 ug/mL; Fab clone #585). Apoptosis was analyzed using the flow cytometry. Treatment with the anti-Wnt16 Fab clone #585 induced apoptosis in 37.3% cells, as compared with 7.92% in cells with a control IgG antibody, respectively.

FIG. 11 shows a Western blot analysis of the Wnt16 protein in different human cell lines using a Wnt16 polyclonal antibody. Cell lines tested include leukemia cell lines 697, NB4, and RCH-ACV; colon cancer cell line SW480; mesothelioma cell line H28; lung cancer cell lines H460, H11703, and A549; and a normal mesothelial cell line, LP9.

FIG. 12 shows Wnt16 expression in non-small cell lung cancer and cell lines analyzed by RT-PCR. A. 697 is a leukemia cell line known to express Wnt16 (positive control); N and T represent one pair of normal (N) and tumor (T) samples originating from the same patient. “*” indicates samples showing the expected PCR fragment indicative for Wnt16 expression. B. Wnt16 expression in the lung cancer cell line H460 and in the breast cancer cell line MCF7. Molecular weigh marker in A and B is the 1-kb plus marker (Invitrogen).

### DEFINITIONS

The terms “Wnt protein” or “Wnt ligand” refer to a family of mammalian proteins related to the Drosophila segment polarity gene, wingless. In humans, the Wnt family of genes typically encode 38 to 43 kDa cysteine rich glycoproteins having hydrophobic signal sequence, and a conserved asparagine-linked oligosaccharide consensus sequence (Shimizu, H. et al., Cell Growth Differ 8(12):1349-58 (1997)). The Wnt family contains at least 21 mammalian members. Exemplary Wnt proteins include Wnt1, Wnt2, Wnt3, Wnt3A, Wnt4, Wnt5A, Wnt5B, Wnt6, Wnt7A, Wnt7B, Wnt8A, Wnt8B, WNT10A, Wnt10B, Wnt11, Wnt12, Wnt13, Wnt14, Wnt15, Wnt16a, Wnt16b, and Wnt16c. Preferred Wnt proteins of the invention are Wnt16a, Wnt16b, and Wnt16c, preferably human Wnt16a, Wnt16b, and Wnt16c, the sequence of which are set forth in the sequence listing. Unless specified otherwise, the term “Wnt16” includes Wnt16 isoforms described herein.

The terms “frizzled protein” or “frizzled receptor” refer to a family of mammalian proteins related to the Drosophila frizzled genes, which play a role in the development of tissue polarity. The Frizzled family comprises at least 10 mammalian genes. Exemplary human Frizzled receptors include Frizzled1, Frizzled2, Frizzled3, Frizzled4, Frizzled5, Frizzled6, Frizzled7, Frizzled8, Frizzled9 and Frizzled10. The mammalian homologues of the Drosophila frizzled protein share a number of common structural motifs. The N terminus located at the extracellular membrane surface is followed by a signal sequence, a domain of 120 amino acids with an invariant pattern of 10 cysteine residues, and a highly divergent region of 40-100 largely variable hydrophilic amino acids. Putative hydrophobic segments form seven membrane-spanning helices linked by hydrophilic loops, ending with the C terminus located at the intracellular face of the membrane. The cysteine-rich domains (CRDs) and the transmembrane segments are strongly conserved, suggesting a working model in which an extracellular CRD is tethered by a variable linker region to a bundle of seven membrane-spanning helices. Frizzled protein receptors are, therefore, involved in a dynamic model of transmembrane signal transduction analogous to G-protein-coupled receptors with amino-terminal ligand binding domains. For example, Frizzled1, Frizzled2, and Frizzled7 are involved in lung and colorectal cancers, (Sagara et al., Biochem Biophys Res Commun 252(1):117-22 (1998)); Frizzled3 in human cancer cells including lung, cervical and colorectal cancers, (Kirikoshi et al., Int J Oncol 19(4):767-71 (2001)); Frizzled7 in gastric cancer (Kirikoshi et al., Int J Oncol 19(4):767-71 (2001)); Frizzled10 in gastric and colorectal cancer, Kirikoshi et al., Int J Oncol 19(4):767-71 (2001); Terasaki et al., Int J Mol Med 9(2):107-12 (2002).

The terms “Dishevelled” or “Dvl” refer to a member of a family of Dishevelled proteins, the full-length sequences of which typically possess three conserved domains, a DIX domain, present in the Wnt antagonizing protein Axin; a PDZ domain involved in protein-protein interactions, and a DEP domain found in proteins that regulate Rho GTPases. Dvl proteins include, for example, Dvl-1, Dvl-2, and Dvl-3. Nucleic acid and protein Dvl sequence are known from a variety of species, including mouse and human. Exemplary human Dvl-1, Dvl-2, and Dvl-3 protein sequences are available under reference sequences NP_{—}004412, NP-004413, and NM_{—}004414, respectively.

“Inhibitors” of Wnt signaling and in particular Wnt16 signaling refers to compounds that, e.g., bind to Wnt or Frizzled proteins, or partially or totally block Wnt signaling as measured in known assays for Wnt signaling (e.g., measurement of β-catenin levels, or oncogene expression controlled by Tcf and Lef transcription factors). Inhibitors, include modified versions of Wnt or Frizzled proteins, as well as naturally occurring and synthetic ligands, antagonists, agonists, antibodies, small chemical molecules, and the like. Assays for detecting inhibitors of the invention are described in more detail below.

The phrases “cell that overexpresses Wnt16 protein,” “cell that overexpresses Wnt16 mRNA,” cancer cell that overexpresses Wnt16 protein” or “cancer cell that overexpresses Wnt16 mRNA” or grammatical equivalents thereof refer to a cell or cancer cell in which expression of a Wnt16 protein or Wnt16 mRNA is at least about 2 times, usually at least about 5 times the level of expression in a normal cell from the same tissue. Methods for determining the level of expression of a particular gene are well known in the art. Such methods include RT-PCR, use of antibodies against the gene products, and the like.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG. See also, Pierce Catalog and Handbook, 1994-1995 (Pierce Chemical Co., Rockford, Ill.). See also, e.g., Kuby, Immunology, 3^{rd} Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al., (1992) J Immuol 148:1547; Pack and Pluckthun, (1992) Biochemistry 31:1579; Hollinger et al., 1993, supra; Gruber et al., (1994) J Immunol:5368; Zhu et al., (1997) Protein Sci 6:781; Hu et al., (1996) Cancer Res. 56:3055; Adams et al., (1993) Cancer Res. 53:4026; and McCartney et al., (1995) Protein Eng. 8:301.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs.” The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or a “VH” refer to the variable region of an immunoglobulin heavy chain of an antibody, including the heavy chain of an Fv, scFv, dsFv (disulphide-stabilized Fv) or Fab. References to “V_{L}” or a “VL” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The term “fully human antibody” refers to an immunoglobulin comprising human variable regions in addition to human framework and constant regions. Such antibodies can be produced using various techniques known in the art. For example in vitro methods involve use of recombinant libraries of human antibody fragments displayed on bacteriophage (e.g., McCafferty et al., 1990, Nature 348:552-554; Hoogenboom & Winter, J. Mol. Biol. 227:381 (1991); and Marks et al., J. Mol. Biol. 222:581 (1991)), yeast cells (Boder and Wittrup, 1997, Nat Biotechnol 15:553-557), or ribosomes (Hanes and Pluckthun, 1997, Proc Natl Acad Sci USA 94:4937-4942). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 6,150,584, 5,545,807; 5,545,806; 5,569,825; 5,625,126; 5,633,425; 5,661,016, and in the following scientific publications: (e.g., Jakobavits, Adv Drug Deliv Rev. 31:33-42 (1998), Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

“Biological sample” as used herein is a sample of biological tissue or fluid that contains nucleic acids or polypeptides, e.g., of a Wnt protein, polynucleotide or transcript. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, preferably a human, but can also be accomplished by using previously isolated cells (e.g., isolated by another person, at another time, and/or for another purpose), or by performing the methods of the invention in vivo. Archival tissues, having treatment or outcome history, will be particularly useful.

The “level of Wnt16 mRNA” in a biological sample refers to the amount of mRNA transcribed from a Wnt16 gene that is present in a cell or a biological sample. The mRNA generally encodes a functional Wnt16 protein, although mutations may be present that alter or eliminate the function of the encoded protein. A “level of Wnt16 mRNA” need not be quantified, but can simply be detected, e.g., a subjective, visual detection by a human, with or without comparison to a level from a control sample or a level expected of a control sample.

The “level of Wnt16 protein or polypeptide” in a biological sample refers to the amount of polypeptide translated from a Wnt16 mRNA that is present in a cell or biological sample. The polypeptide may or may not have Wnt16 protein function. A “level of Wnt16 protein” need not be quantified, but can simply be detected, e.g., a subjective, visual detection by a human, with or without comparison to a level from a control sample or a level expected of a control sample.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., Altschul et al., Nucl Acids Res 25:3389-34021 (1977) and Altschul et al., J Mol Biol 215:403-410 (1990)). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nucl Acids Res 25:3389-3402 (1977) and Altschul et al., J Mol Biol 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc Natl Acad Sci USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc Natl Acad Sci USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The radioisotope may be, for example, 3H, 14C, 32P, 35S, or 125I. In some cases, particularly using antibodies against the proteins of the invention, the radioisotopes are used as toxic moieties, as described below. The labels may be incorporated into the nucleic acids, proteins and antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982). The lifetime of radiolabeled peptides or radiolabeled antibody compositions may extended by the addition of substances that stabilize the radiolabeled peptide or antibody and protect it from degradation. Any substance or combination of substances that stabilize the radiolabeled peptide or antibody may be used including those substances disclosed in U.S. Pat. No. 5,961,955.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody.

The “effector” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

The phrase “specifically (or selectively) binds” to an antibody or antigen, such as a protein, preferably a Wnt16 protein or “specifically (or selectively) immunoreactive with,” when referring to a protein or peptide or antibody, refers to a binding reaction that is determinative of the presence of the protein, in a heterogeneous population of proteins and other biologics. Thus, under designated immunoassay conditions, the specified antibodies bind to a particular protein at least two times the background and more typically more than 10 to 100 times background.

Specific binding to an antibody under such conditions requires an antibody that is selected for its specificity for a particular protein. For example, antibodies raised to a particular protein, polymorphic variants, alleles, orthologs, and conservatively modified variants, or splice variants, or portions thereof, can be selected to obtain only those antibodies that are specifically immunoreactive with Wnt16 proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

“Tumor cell” refers to precancerous, cancerous, and normal cells in a tumor.

“Cancer cell,” “transformed” cell or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. In the present invention transformation is typically associated with overexpression of Wnt, in particular with Wnt16, and/or Frizzled proteins. Transformation is associated with other phenotypic changes, such as immortalization of cells, aberrant growth control, nonmorphological changes, and/or malignancy (see, Freshney, Culture of Animal Cells a Manual of Basic Technique (3rd ed. 1994)).

By “small interfering RNA” or “siRNA” is meant an isolated RNA molecule, preferably greater than 10 nucleotides in length, more preferably greater than 15 nucleotides in length, and most preferably 18, 19, 20, 21, 22, 23, 24, 25, 26, 27, 28, 29, or 30 nucleotides in length that has been shown to function as a key intermediate in triggering sequence-specific RNA degradation. A range of 19-25 nucleotides is the most preferred size for siRNAs. siRNAs can also include short hairpin RNAs (shRNA) in which both strands of an siRNA duplex are included within a single RNA molecule. Double-stranded siRNAs generally consist of a sense and anti-sense strand. Single-stranded siRNAs generally consist of only the antisense strand that is complementary to the target gene or mRNA. siRNA includes any form of RNA, preferably dsRNA (proteolytically cleaved products of larger dsRNA, partially purified RNA, essentially pure RNA, synthetic RNA, recombinantly produced RNA) as well as modified RNA that differs from naturally occurring RNA by the addition, deletion, substitution, and/or alteration of one or more nucleotides.

### DETAILED DESCRIPTION

The role of Wnt-Fz signaling pathway in oncogenesis has been described to some extent in WO 04/032838. The present invention provides inhibitors of Wnt16 signaling pathway that can induce significant apoptosis in a number of cells and cancers overexpressing Wnt16. The invention is useful for treatment of a disease associated with Wnt16 signaling, in particular a cancer in which Wnt16 signaling, particularly Wnt16b signaling, affects cancer cell growth or survival. The invention is particularly useful for treating cancers such as acute lymphoblastoid leukemia (ALL), pre-B acute lymphoblastoid leukemia (pre-B ALL) and B cell chronic lymphocytic leukemia (CLL) as well as lung cancer, mesothelioma, melanoma, colon cancer, brain cancer, breast cancer, kidney cancer, leukemia and lymphoma. The invention is also useful for treating a disease associated with abnormal or atypical Wnt16 signaling in the placenta, heart or spleen.

### I. Antibodies to Wnt16 Proteins

As noted above, the invention provides methods of inhibiting Wnt16 signaling in cells overexpressing Wnt16, preferably cancer cells. In some embodiments of the invention, antibodies are used to block the binding between Wnt16 ligand and the Frizzled receptor. The antibodies can be raised against either a Wnt or a Frizzled receptor protein. Preferred are anti-Wnt16 antibodies. Production of antibodies useful in the invention are described, for example, in WO 04/032838.

A. Generation of Anti-Wnt 16 Monoclonal Antibodies

Antibodies that may be used in the methods, pharmaceutical compositions and kits of the present invention maybe polyclonal anti-Wnt16 antibodies, monoclonal anti-Wnt16 antibodies or anti-Wnt16 Fabs as fully described herein. Preferably, the antibodies are monoclonal anti-Wnt16 antibodies. Monoclonal antibodies of the invention may be prepared in a variety of ways. A preferred method uses hybridoma methods, such as those described by Kbhler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

B. Antigenic Wnt16 Polypeptides

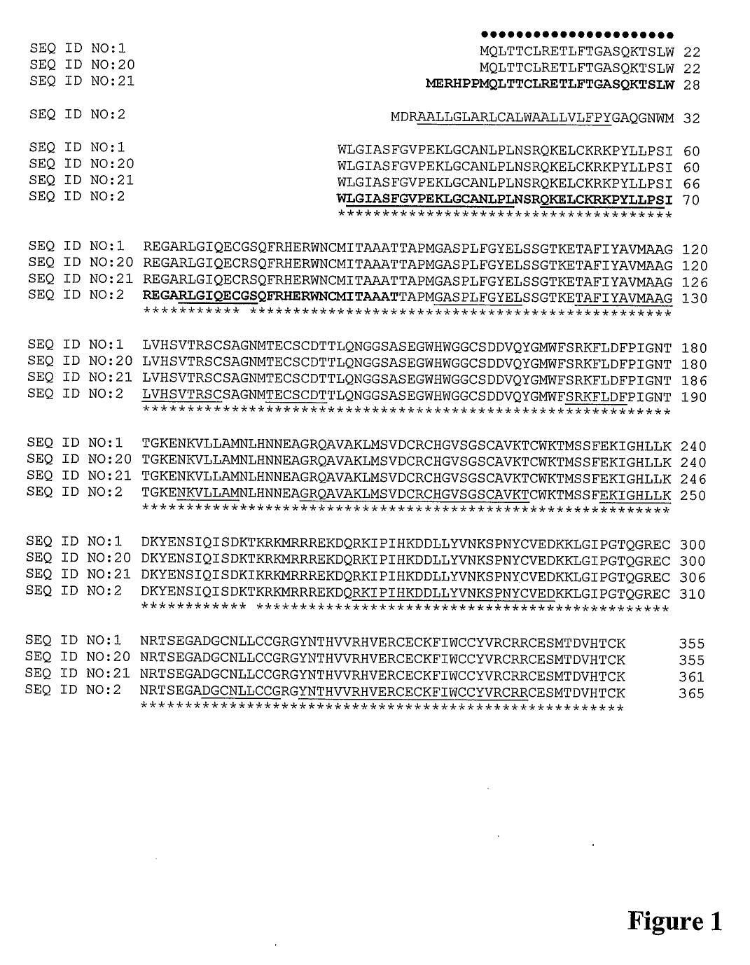

Several anti-Wnt16 monoclonal antibodies can be generated using the methods and immunizing agents described herein. In one aspect of the present invention, the immunizing agent will typically include a Wnt16 polypeptide, for example, a Wnt16 isoform as shown in SEQ ID NO:1 (Wnt16a) or SEQ ID NO:2 (Wnt16b). Other useful Wnt16 isoforms for use as immunizing agent are shown in SEQ ID NO:20 and SEQ ID NO 21. Wnt16 protein isoforms are described as consisting of either 355 amino acid residues (Wnt16a; Gen Bank accession numbers EAL24346 and NP_{—}057171 (SEQ ID NO:1)), 365 amino acid residues (Wnt16b; GenBank accession numbers NP 476509 (SEQ ID NO: 2); Q9UBV4, EAL24346 and AAD49351) or 361 amino acid residues (Wnt16c; GenBank accession number AAD38052). These isoforms differ mainly at their N-terminus (FIG. 1). Wnt16a shows the specific amino acid sequence MQLTTCLRETLFTGASQKTSLW (SEQ ID NO:22; FIG. 1) at its N-terminus. Wnt16c further includes, in addition to SEQ ID NO:22, the sequence MERHPP (SEQ ID NO:23; FIG. 1) at its N-terminus. Wnt16b shows a different sequence at its N-terminus: MDRAALLGLARLCALWAALLVLFPYGAQGNWM (SEQ ID NO:24; FIG. 1). However, there are also two variable internal positions: Wnt16a has either a glycine (G) at position 72 (see, SEQ ID NO:1 and FIG. 1) or an arginine (R; see, SEQ ID NO:20 and FIG. 1). At the corresponding position, Wnt16b has a glycine (G; see, SEQ ID NO:2 and FIG. 1) and Wnt16c has an arginine (R; see, SEQ ID NO:21). Another variable position occurs at position 253 of Wnt16a, and the corresponding position 263 in Wnt16b and 259 of Wnt16c. At this position, Wnt16a and Wnt16b have a threonine (T; see, SEQ ID NOS:1, 20 and 2; FIG. 1), Wnt16c reveals an isoleucine (I; see SEQ ID NO: 21; FIG. 1).

Thus, in another aspect of the present invention, an anti-Wnt16 antibody is generated using as an immunizing peptide a peptide that is specific for a particular Wnt16 isoform, such as peptides shown in SEQ ID NOS: 22, 23, and 24. A preferred antigenic peptide comprises amino acid residues 1-99 of human Wnt16 as shown in SEQ ID NO:2 (FIG. 1). Antigenic peptides may be generated synthetically or may be expressed in vivo, e.g., in E. coli. For example a polypeptide comprising amino acid residues 1-99 of human Wnt16 as shown in SEQ ID NO:2 (FIG. 1) was expressed in E. coli, purified and used as an antigen.

In another aspect of the present invention, the immunizing agent is an antigenic peptide of human Wnt16. Antigenic peptides of human Wnt16 protein can be determined using various methods. For example, the EMBOSS method (Parker et al., Biochemistry 25:5425-5432 (1986)) finds antigenic sites in proteins. Antigenic peptides can also determined using the method of Kolaskar and Tangaonkar (K&T; FEBS Lett. (1990) 276(1-2):172-4). Both methods lead to the identification of similar antigenic peptides of human Wnt16 (Table 1). While most of the antigenic peptide sequences identified can be used to generate antibodies that specifically bind to human Wnt16, some antibodies may bind to Wnt16a, Wnt16b and/or Wnt16c proteins due to amino acid sequence homology among these Wnt16 isoforms.

TABLE 1
Antigenic Peptides of Human Wnt16b

| | | Antigenic peptide |
|---|---|---|
| Sequence of Antigenic Peptide of Human Wnt16b | | present also in |
| and Position within SEQ ID NO:2 | SEQ ID NO | SEQ ID NOS |
| 4 AALLGLARLCALWAALLVLFPY 25 | 3 | |
| 56 QKELCKRKPYLLPS 69 | 4 | 1, 20, 21 |
| 75 RLGIQECGS 83 | 5 | 1, 21 |
| 104 GASPLFGYEL 113 | 6 | 1, 20, 21 |
| 120 TAFIYAVMAAGLVHSVTRSC 139 | 7 | 1, 20, 21 |
| 145 TECSCDT 151 | 8 | 1, 20, 21 |
| 179 SRKFLDF 185 | 9 | 1, 20, 21 |
| 195 NKVLLAM 201 | 10 | 1, 20, 21 |
| 209 GRQAVAKLMSVDCRCHGVSGSCAVKT 234 | 11 | 1, 20, 21 |
| 243 EKIGHLLK 250 | 12 | 1, 20, 21 |
| 275 RKIPIHKDDLLYVNKSPNYCVED 297 | 13 | 1, 20, 21 |
| 318 DGCNLLCCG 326 | 14 | 1, 20, 21 |
| 329 YNTHVVRHVERCECKFIWCCYVRCRR 354 | 15 | 1, 20, 21 |
| 34 LGIASFGVPEKLGCANLPL 52 | 16 | 1, 20, 21 |

C. Anti-Wnt16 Antibodies

Antigenic peptides disclosed herein can be used to generate polyclonal and monoclonal anti-Wnt16 antibodies. Preferred are monoclonal antibodies.

In a preferred embodiment of the present invention, an anti-Wnt16 antibody is antibody that specifically binds a polypeptide comprising an amino acid sequence as shown in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16. Also preferred is an anti-Wnt16 antibody that specifically binds a polypeptide comprising an amino acid sequence as shown in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16, wherein the polypeptide consists of an amino acid sequence that is different from SEQ ID NO:1, SEQ ID NO:2, SEQ ID NO:20 and SEQ ID NO:21 and is also different from the sequence of a mature Wnt16 protein, i.e., a Wnt16 protein from which the signal peptide has been cleaved off.

In yet another preferred embodiment of the present invention, an anti-Wnt16 antibody is an antibody that specifically binds a polypeptide comprising amino acid residues 1-99 of Wnt16 as shown in SEQ ID NO:2. Also preferred is an anti-Wnt16 antibody that specifically binds a polypeptide comprising an amino acid sequence corresponding to amino acid residues 1-99 of human Wnt16 as shown in SEQ ID NO:2. Preferably, this antibody is a human antibody and even more preferably a human Fab.

In another preferred embodiment of the present invention, an anti-Wnt16 antibody is an antibody that specifically binds a polypeptide consisting of amino acid residues 1-99 of Wnt16 as shown in SEQ ID NO:2. Also preferred is an anti-Wnt16 antibody that specifically binds a polypeptide consisting of an amino acid sequence corresponding to amino acid residues 1-99 of human Wnt16 as shown in SEQ ID NO:2. Preferably, this antibody is a human antibody and even more preferably a human Fab.

In one embodiment of the present invention an anti-Wnt16 antibody specifically binds to a Wnt16 protein or Wnt16 peptide. In another embodiment of the invention an anti-Wnt16 antibody of the present invention binds to a cell surface antigen of a Wnt16 expressing cell. In yet another embodiment of the present invention, an anti-Wnt16 antibody inhibits binding of a Wnt16 protein to a receptor, such as a Frizzled receptor. In another preferred embodiment of the present invention, a Wnt16 antibody inhibits Wnt16 signaling.

D. Generation of Recombinant Anti-Wnt16 Antibodies

Anti-Wnt16 antibodies of the present invention can be prepared in a variety of ways. In one aspect of the present invention, anti-Wnt16 antibodies are produced recombinantly. In this method, the nucleic acid encoding an anti-Wnt16 monoclonal antibody produced by a hybridoma cell is sequenced. Preferably, the nucleic acid encoding the complementary determining region (CDR) of the monoclonal antibody is sequenced. Thus, in one aspect of the invention an anti-Wnt16 antibody is produced by inserting a nucleic acid encoding the anti-Wnt16 antibody into an expression vector, which is then transformed or transfected into an appropriate host cell. The host cell is then cultivated under conditions suitable for expression.

These procedures are generally known in the art, as described generally in Maniatis et al., Molecular Cloning, A Laboratory Manual, Cold Spring Harbor Press, Cold Spring Harbor, N.Y. (1982), which is incorporated herein by reference. “Vector” refers to any type of genetic construct containing a nucleic acid capable of being transcribed in a cell. Vectors used for the amplification of nucleotide sequences (both coding and non-coding) are also encompassed by the definition. In addition to the coding sequence, vectors will generally include restriction enzyme cleavage sites and the other initial, terminal and intermediate DNA sequences that are usually employed in vectors to facilitate their construction and use. The expression vector can be part of a plasmid, virus, or nucleic acid fragment. “Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-o-methyl ribonucleotides and peptide-nucleic acids (PNAs).

Coding sequences for the anti-Wnt16 monoclonal antibodies of the present invention or fragments and CDR sequences thereof may be synthesized by chemical techniques, for example, the phosphotriester method of Matteucci et al. (J Am Chem Soc. 1981, 103:3185). The term “coding sequence”, in relation to nucleic acid sequences, refers to a plurality of contiguous sets of three nucleotides, termed codons, each codon corresponding to an amino acid as translated by biochemical factors according to the universal genetic code, the entire sequence coding for an expressed protein, or an antisense strand that inhibits expression of a protein. A “genetic coding sequence” is a coding sequence where the contiguous codons are intermittently interrupted by non-coding intervening sequences, or “introns.” During mRNA processing intron sequences are removed, restoring the contiguous codon sequence encoding the protein.

Any modification within a DNA or RNA sequence can be made simply by substituting the appropriate bases for those encoding the desired amino acid sequence. The coding sequence can then be provided with appropriate linkers and ligated into expression vectors commonly available in the art, and the vectors used to transform suitable hosts to produce the immunostimulating peptide or protein. A number of such vectors and suitable host systems are commercially available. For expression, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast or mammalian cell hosts may also be used, employing suitable vectors and control sequences as known to the skilled artisan.

E. Chimeric and Humanized Anti-Wnt16 Antibodies

In some embodiments of the invention the anti-Wnt16 antibodies are chimeric or humanized antibodies. As noted above, humanized forms of antibodies are chimeric immunoglobulins in which residues from a complementary determining region (CDR) of human antibody are replaced by residues from a CDR of a non-human species such as mouse, rat or rabbit having the desired specificity, affinity and capacity.

Human antibodies can be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

F. Single Chain Fv Antibodies Binding Wnt16

In some embodiments, the antibody is a single chain Fv (scFv). The V_{H} and the V_{L} regions of a scFv antibody comprise a single chain which is folded to create an antigen binding site similar to that found in two chain antibodies. Once folded, noncovalent interactions stabilize the single chain antibody. While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993). Generally the peptide linker will have no specific biological activity other than to join the regions or to preserve some minimum distance or other spatial relationship between the V_{H} and V_{L}. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatamer of the sequence Gly-Gly-Gly-Gly-Ser, preferably 2, 3, 4, 5, or 6 such sequences. However, it is to be appreciated that some amino acid substitutions within the linker can be made. For example, a valine can be substituted for a glycine.

Methods of making scFv antibodies have been described. See, Huse et al, supra; Ward et al. supra; and Vaughan et al, supra. In brief, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell. The scFv that specifically bind to the desired antigen are typically found by panning of a phage display library. Panning can be performed by any of several methods. Panning can conveniently be performed using cells expressing the desired antigen on their surface or using a solid surface coated with the desired antigen. Conveniently, the surface can be a magnetic bead. The unbound phage are washed off the solid surface and the bound phage are eluted.

Regardless of the method of panning chosen, the physical link between genotype and phenotype provided by phage display makes it possible to test every member of a cDNA library for binding to antigen, even with large libraries of clones.

G. Bispecific and Conjugated Anti-Wnt16 Antibodies

In some embodiments, the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for the Wnt16 protein, the other one is for another protein, for example a cancer antigen. Alternatively, tetramer-type technology may create multivalent reagents.

In some embodiments, the antibody is conjugated to an effector moiety. The effector moiety can be any number of molecules, including labeling moieties such as radioactive labels or fluorescent labels, or can be a therapeutic moiety. If the effector moiety is a therapeutic moiety, it will typically be a cytotoxic agent. In this method, targeting the cytotoxic agent to cancer cells, results in direct killing of the target cell. This embodiment is typically carried out using antibodies against the Frizzled receptor. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, auristatin and the like. Cytotoxic agents also include radiochemicals made by conjugating radioisotopes to antibodies raised against Wnt16 proteins, or binding of a radionuclide to a chelating agent that has been covalently attached to the antibody.

H. Binding Affinity of Antibodies of the Invention

Binding affinity for a target antigen is typically measured or determined by standard antibody-antigen assays, such as Biacore competitive assays, saturation assays, or immunoassays such as ELISA or RIA.

Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is <1 μM, preferably <100 nM, and most preferably <0.1 nM. Antibody molecules will typically have a K_{D} in the lower ranges. K_{D}=[Ab-−Ag]/[Ab][Ag] where [Ab] is the concentration at equilibrium of the antibody, [Ag] is the concentration at equilibrium of the antigen and [Ab−Ag] is the concentration at equilibrium of the antibody-antigen complex. Typically, the binding interactions between antigen and antibody include reversible noncovalent associations such as electrostatic attraction, Van der Waals forces and hydrogen bonds.

The antibodies of the invention specifically bind to Wnt16 proteins. By “specifically bind” herein is meant that the antibodies bind to the Wnt16 protein with a K_{D} of at least about 0.1 mM, more usually at least about 1 μM, preferably at least about 0.1 μM or better, and most preferably, 0.01 μM or better.

I. Competitive Binding of Anti-Wnt16 Monoclonal Antibodies

In some embodiments, an anti-Wnt16 monoclonal antibody is used. A preferred embodiment is an anti-Wnt16 monoclonal antibody that binds the same epitope as a second anti-Wnt16 antibody, for example, the anti-Wnt16 monoclonal antibody described in the Examples, below. The ability of a particular antibody to recognize the same epitope as another antibody is typically determined by the ability of one antibody to competitively inhibit binding of the second antibody to the antigen. Any of a number of competitive binding assays can be used to measure competition between two antibodies to the same antigen. For example, a sandwich ELISA assay can be used for this purpose. This is carried out by using a capture antibody to coat the surface of a well. A subsaturating concentration of tagged-antigen is then added to the capture surface. This protein will be bound to the antibody through a specific antibody:epitope interaction. After washing a second antibody, which has been covalently linked to a detectable moiety (e.g., HRP, with the labeled antibody being defined as the detection antibody) is added to the ELISA. If this antibody recognizes the same epitope as the capture antibody it will be unable to bind to the target protein as that particular epitope will no longer be available for binding. If, however, this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

In a preferred embodiment of the present invention, the anti-Wnt16 antibody is an anti-Wnt16 antibody that competes for binding a Wnt16 protein with a second anti-Wnt16 antibody that specifically binds a polypeptide comprising an amino acid sequence as shown in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16. Also preferred is an anti-Wnt16 antibody that competes for binding a Wnt16 protein with a second anti-Wnt16 antibody that specifically binds a polypeptide comprising an amino acid sequence as shown in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16, wherein the polypeptide consists of an amino acid sequence that is different from SEQ ID NO:1, SEQ ID NO:2, SEQ ID NO:20 and SEQ ID NO:21 and is also different from the sequence of a mature Wnt16 protein, i.e., a Wnt16 protein from which the signal peptide has been cleaved off.

In yet another preferred embodiment of the present invention, an anti-Wnt16 antibody competes for binding a Wnt16 protein with a second anti-Wnt16 antibody that specifically binds a polypeptide comprising amino acid residues 1-99 of Wnt16 as shown in SEQ ID NO:2. Also preferred is an anti-Wnt16 antibody that competes for binding a Wnt16 protein with a second anti-Wnt16 antibody that specifically binds a polypeptide comprising an amino acid sequence corresponding to amino acid residues 1-99 of human Wnt16 as shown in SEQ ID NO:2. Preferably, the anti-Wnt16 antibody competing with a second Wnt16 antibody is a human antibody and even more preferably a human Fab.

In another preferred embodiment of the present invention, an anti-Wnt16 antibody competes for binding a Wnt16 protein with a second anti-Wnt16 antibody that specifically binds a polypeptide consisting of amino acid residues 1-99 of Wnt16 as shown in SEQ ID NO:2. Also preferred is an anti-Wnt16 antibody that competes for binding a Wnt16 protein with a second anti-Wnt16 antibody that specifically binds a polypeptide consisting of an amino acid sequence corresponding to amino acid residues 1-99 of human Wnt16 as shown in SEQ ID NO:2. Preferably, the anti-Wnt16 antibody competing with a second Wnt16 antibody is a human antibody and even more preferably a human Fab.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

### II. Assays for Detecting Levels of Wnt16 Expression

The present invention also provides diagnostic assays for detecting Wnt16. In preferred embodiments, activity of the Wnt16 gene is determined by a measure of gene transcript (e.g. mRNA), by a measure of the quantity of translated protein, or by a measure of gene product activity.

Methods of detecting and/or quantifying the gene transcript (mRNA or cDNA) using nucleic acid hybridization techniques are known to those of skill in the art. For example, one method for evaluating the presence, absence, or quantity of mRNA involves a Northern blot transfer.

The probes can be full length or less than the full length of the nucleic acid sequence encoding the Wnt16 protein. Probes usually are labeled with, for example, with a radionucleotide or biotin and can be generated by nick translation, random or specific priming as known in the art. Hybridization conditions are also described in the art. These procedures are generally known in the art, as described generally in Maniatis et al., Molecular Cloning, A Laboratory Manual, Cold Spring Harbor Press, Cold Spring Harbor, N.Y. (1982). Shorter probes are empirically tested for specificity. Specific Wnt16 isoform mRNAs, i.e., a Wnt16a, Wnt16b or Wnt16 c mRNA or a Wnt mRNA encoding a Wnt16 protein as shown in SEQ ID NO:1, SEQ ID NO:2, SEQ ID NO:20 or SEQ ID NO:21 can be detected using methods described in the prior art. These methods can be used to discriminate nucleic acids differing by a single base pair mismatch (Wallace et al., Nucleic Acids Res 9(4):879-94 (1991); Conner et al., Proc Natl Acad Sci USA 80(1):278-82 (1983)). Preferably nucleic acid probes are 20 bases or longer in length Visualization of the hybridized portions allows the qualitative determination of the presence or absence of mRNA.

In another preferred embodiment, a transcript (e.g., mRNA) can be measured using amplification (e.g. PCR) based methods as described above for directly assessing copy number of DNA or mRNA. In a preferred embodiment, transcript level is assessed by using reverse transcription PCR (RT-PCR). Primer pairs useful in such methods are disclosed in SEQ ID NOS: 17-19.

The “activity” of the Wnt16 gene can also be detected and/or quantified by detecting or quantifying the expressed Wnt16 polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like. The isolated proteins can also be sequenced according to standard techniques to identify polymorphisms.

The antibodies of the invention can also be used to detect the Wnt16 protein, or cells expressing them, using any of a number of well recognized immunological binding assays (see, e.g., U.S. Pat. Nos. 4,366,241; 4,376,110; 4,517,288; and 4,837,168). For a review of the general immunoassays, see also Methods in Cell Biology, Vol. 37, Asai, ed. Academic Press, Inc. New York (1993); Basic and Clinical Immunology 7th Edition, Stites & Terr, eds. (1991).

Thus, the present invention provides methods of detecting cells, that over-express Wnt16 protein, in particular cancer cells. Typically, Wnt16 expression is analyzed in a biological sample. In one method, a biopsy is performed on the subject and the collected tissue is tested in vitro. The tissue or cells from the tissue is then contacted, with an anti-Wnt16 antibody of the invention. An immune complex which results indicates the presence of a Wnt16 protein in the biopsied sample. To facilitate such detection, the antibody can be radiolabeled or coupled to an effector molecule which is a detectable label, such as a radiolabel.

In another method, the cell or cancer cell overexpressing Wnt16 is detected in vivo using, for example, typical imaging systems. Then, the localization of the label is determined by any of the known methods for detecting the label. A conventional method for visualizing diagnostic imaging can be used. For example, paramagnetic isotopes can be used for MRI. Internalization of the antibody may be important to extend the life within the organism beyond that provided by extracellular binding, which will be susceptible to clearance by the extracellular enzymatic environment coupled with circulatory clearance.

The methods described above can also be used in prognostic assays or to predict drug response, that is as a pharmacogenomic marker. In particular, the methods can be used to predict a response to therapeutic regimens described herein. For example, such methods can be used to predict a response to therapeutic methods using the anti-Wnt16 antibodies of the invention.

III. Methods Using Anti-Wnt16 Antibodies and siRNA

Agents that inhibit Wnt16 signaling, such as the anti-Wnt16 antibodies and siRNAs of the invention may find use in a variety of ways. In a preferred embodiment of this invention a method of inhibiting proliferation of a cell that overexpresses a Wnt16 is provided. The Wnt16 that is overexpressed can be either a Wnt16 protein or a Wnt16 mRNA. This method comprises the step of contacting the cell with an amount of an agent that inhibits Wnt16 signaling effective to inhibit proliferation of the cell. “Proliferation” refers to the growth of a cell, the reproduction or multiplication of a cell or morbid cysts.

In a preferred embodiment of the present invention, this method is practiced in vitro. As further described herein, the methods of the present invention can also be practiced in vivo.

In a preferred embodiment of the present invention, the cell being contacted with the agent is a cancer cell. Agents of the present invention are useful for inhibiting proliferation of a cancer cell selected from the group consisting of acute lymphoblastoid leukemia (ALL) cell, pre-B acute lymphoblastoid leukemia (pre-B ALL) cell, B cell chronic lymphocytic leukemia (CLL) cell, lung cancer cell, mesothelioma cell, melanoma cell, colon cancer cell, brain cancer cell, breast cancer cell, kidney cancer cell, leukemia cell and lymphoma cell. In one embodiment of the present invention, the cancer cell is a lung cancer cell. In another embodiment of the present invention, the cancer cell is an ALL cell. In another preferred embodiment, the cancer cell is a pre-B ALL cell. In yet another preferred embodiment of the present invention, the cancer cell is a CLL cell. The invention is also useful for treating a disease associated with abnormal or atypical Wnt16 signaling in the placenta, heart or spleen.

A. Inducing Apoptosis

Agents that inhibit Wnt16 signaling, such as the anti-Wnt16 antibodies and siRNAs of the invention may find use in a variety of ways. In another preferred embodiment of this invention a method of inducing apoptosis of a cell that overexpresses a Wnt16 is provided. This method comprises the step of contacting the cell with an amount of an agent that inhibits Wnt16 signaling effective to induce apoptosis of the cell. Agents for use in this method, such as anti-Wnt16 antibodies or siRNAs are disclosed herein.

B. Inhibiting Wnt16 Signaling

Agents of the present invention, such as the anti-Wnt16 antibodies and siRNAs of the invention may find use in a variety of ways. In another preferred embodiment of this invention a method of inhibiting Wnt16 signaling in a cell is provided. This method comprises the step of contacting a cell that overexpresses a Wnt16 with an amount of an agent effective to inhibit Wnt16 signaling. Agents for use in this method, such as anti-Wnt16 antibodies or siRNAs are disclosed herein.

C. Treating a Disease

Agents of the present invention, such as the anti-Wnt16 antibodies and siRNAs of the invention may find use in a variety of ways. In a preferred embodiment of this invention a method of treating a disease associated with Wnt16 signaling is provided. This method comprises the step of administering to a subject, preferably to a subject in need of such treatment, an amount of an agent that inhibits Wnt16 signaling effective to treat the disease. Preferably, the subject is a human. Agents for use in this method, such as anti-Wnt16 antibodies or siRNAs are disclosed herein.

In a preferred embodiment the disease is a cancer. Agents of the present invention are useful for treating a cancer selected from the group consisting of acute [or lymphoblastoid leukemia (ALL), pre-B acute lymphoblastoid leukemia (pre-B ALL), B cell chronic lymphocytic leukemia (CLL), lung cancer, mesothelioma, melanoma, colon cancer, brain cancer, breast cancer, kidney cancer, leukemia and lymphoma. In one embodiment of the present invention, the cancer is a lung cancer. In another embodiment of the present invention, the cancer is ALL. In another preferred embodiment, the cancer is a pre-B ALL. In yet another preferred embodiment of the present invention, the cancer is CLL.

The invention is also useful for treating a disease associated with abnormal or atypical Wnt16 signaling in the placenta, heart or spleen.

As used herein, the terms “treat”, “treating”, and “treatment” include: (1) preventing a disease, such as cancer, i.e. causing the clinical symptoms of the disease not to develop in a subject that may be predisposed to the disease but does not yet experience any symptoms of the disease; (2) inhibiting the disease, i.e. arresting or reducing the development of the disease or its clinical symptoms; or (3) relieving the disease, i.e. causing regression of the disease or its clinical symptoms. Preferably, the subject in need of such treatment is a mammal, more preferable a human.

This invention also provides a method of treating a cancer that overexpresses Wnt16. This method comprises the step of administering to a subject in need of such treatment an amount of an agent effective to treat the cancer. Agents for use in this method, such as anti-Wnt2 antibodies or siRNAs are disclosed herein.

D. Detecting Cancer Cells in a Subject

Agents of the present invention, such as the anti-Wnt16 antibodies and siRNAs of the invention may find use in a variety of ways. In a preferred embodiment of this invention a method of detecting a cancer cell in a subject is provided. This method comprises the steps of providing a biological sample from the subject, wherein the biological sample comprises a cell suspected of being a cancer cell and detecting the level of Wnt16 expression in the cell. Optionally, this method comprises comparing the level of Wnt16 expression in the cell with the level of Wnt16 expression in a cell from one or more healthy subjects or with a previously determined reference range for a level of Wnt16 expression. In one embodiment of the invention, detecting the level of Wnt16 expression is carried out by detecting the level of Wnt16 mRNA. In another embodiment of the invention, detecting the level of Wnt16 expression is carried out by detecting the level of Wnt16 protein. Agents for use in this method, such as anti-Wnt16 antibodies or siRNAs are disclosed herein.

Detection of the level of Wnt16 expression may be determined for a variety of reasons. Detecting the level of Wnt16 expression may be (i) part of screening, diagnosis or prognosis of cancer in the subject; (ii) part of determining susceptibility of the subject to cancer; (iii) part of determining the stage or severity of a cancer in the subject; (iv) part of identifying a risk for the subject of developing a cancer; or (v) part of monitoring the effect of an anti-cancer drug or therapy administered to the subject diagnosed with cancer. The anti-cancer drug or therapy administered to the subject may comprise an anti-Wnt2 antibody or a siRNA of this invention.

In a preferred embodiment of this invention a method for identifying in a subject the stage or severity of a cancer, is provided. As shown herein, Wnt16 expression is overexpressed in various cancer cells, such as leukemia cells, lung cancer cells and breast cancer cells. As further shown herein, anti Wnt16 antibodies and siRNA induce in a dose-dependent manner apoptosis in those cells. Thus, amounts of Wnt16 are characteristic of various cancer risk states, e.g., high, medium or low. The stage or severity of a cancer may be determined by measuring Wnt16 and then either submitting them to a classification algorithm or comparing them with a reference amount and/or pattern of Wnt16 that is associated with a particular stage or severity of the cancer.

Using the methods of the invention, Wnt16 levels are determined in a biological sample from a subject for whom a risk of developing cancer is to be determined. A Wnt16 level detected in a biological sample from the subject for whom a risk of developing cancer is to be determined that is higher than the Wnt16 level detected in a comparable biological sample from normal or healthy subjects or lower than a predetermined base level, indicates that the subject for whom a risk of developing cancer is to be determined has a risk of developing cancer.

In another preferred embodiment of the present invention, a cancer in a subject is determined as part of screening, diagnosis or prognosis of the cancer in the subject. Using the methods of the invention, Wnt16 levels are determined in a biological sample from a subject to be screened for cancer. A Wnt16 level detected in a biological sample from the subject to be screened for cancer that is higher than the Wnt16 level detected in a comparable biological sample from normal or healthy subjects or higher than a predetermined base level, indicates that the subject screened for cancer has or is likely to have cancer.

As described above, Wnt16 compositions are useful for treatment of cancer wherein Wnt16 expression is overexpressed. However, other drugs, for example, a composition comprising an inhibitor of Wnt16, as described herein, will also be useful for treating a cancer in a patient wherein Wnt16 expression is overexpressed. Thus, in a preferred embodiment of the present invention, a cancer status is determined as part of monitoring the effect of surgery (e.g., removal of tumor), the effect of an anti-cancer drug or a therapy administered to a subject diagnosed with a cancer wherein Wnt16 expression is overexpressed. The effect of surgery or an anti-cancer drug or a therapy administered to a subject with cancer may include reoccurrence of cancer, progression of cancer (worsening) and cancer regression (improvement).

Using the compositions, methods and kits of the present invention, Wnt16 levels are determined in a biological sample from a subject at various times after surgery or at various times of having been given an anti-cancer drug or a therapy. A Wnt16 level detected in a biological sample from a subject at a first time (t1; e.g., before giving an anti-cancer drug or a therapy) that is higher than the Wnt16 level detected in a comparable biological sample from the same subject taken at a second time (t2; e.g., after giving the anti-cancer drug or the therapy), indicates that the cancer in the subject is regressing. Likewise, a higher Wnt16 level at a second time compared to a Wnt16 level at a first time, indicates that the cancer in the subject is progressing. Similarly, a Wnt16 level detected in a biological sample from a subject at a first time (t1; e.g., shortly after surgery) that is higher than the Wnt16 level detected in a comparable biological sample from the same subject taken at a second time (t2; e.g., weeks or months after surgery), may indicate that the cancer in the subject is not reoccurring. Likewise, a higher Wnt16 level at the second time compared to the Wnt16 level at the first time, may indicate that the cancer in the subject is reoccurring.

E. siRNA for Use in the Methods of the Invention

Agents of the present invention that are useful for practicing the methods of the present invention include, but are not limited to anti-Wnt16 antibodies and siRNAs of Wnt16. Typically, such agents are capable of (i) binding to Wnt16 mRNA or Wnt16 protein, (ii) interfere with Wnt16 signaling and/or (iii) inhibit binding of Wnt16 protein to other proteins, such as a Frizzled receptor. In a preferred embodiment, the agent inhibiting cell proliferation is a siRNA of Wnt16. The present invention provides compositions and methods using RNA interference to modulate Wnt16 expression. These methods and compositions are useful for the treatment of disease, in particular cancer, induction of apoptosis and interfering with Wnt16 signaling.

In many species, introduction of double-stranded RNA (dsRNA) which may alternatively be referred to herein as small interfering RNA (siRNA), induces potent and specific gene silencing, a phenomena called RNA interference or RNAi. This phenomenon has been extensively documented in the nematode C. elegans (Fire et al., Nature, 391, 806-811, 1998), but is widespread in other organisms, ranging from trypanasomes to mouse. Depending on the organism being discussed, RNA interference has been referred to as “cosuppression”, “post-transcriptional gene silencing”, “sense suppression” and “quelling.” RNAi is an attractive biotechnological tool because it provides a means for knocking out the activity of specific genes. It is particularly useful for knocking out gene expression in species that were not previously considered to be amenable to genetic analysis or manipulation.

RNAi is usually described as a post-transcriptional gene-silencing (PTGS) phenomenon in which dsRNAs trigger degradation of homologous mRNA in the cytoplasm. The basic process involves a dsRNA that is processed into shorter units (called short interfering RNAs (siRNAs)) that guide recognition and targeted cleavage of homologous messenger RNA (mRNA). The dsRNAs that (after processing) trigger RNAi/PTGS can be made in the nucleus or cytoplasm in a number of ways. The processing of dsRNA into siRNAs, which in turn degrade mRNA, is a two-step RNA degradation process. The first step involves a dsRNA endonuclease (ribonuclease III-like; RNase III-like) activity that processes dsRNA into sense and antisense RNAs which are 21 to 25 nucleotides (nt) long (i.e., siRNA). In Drosophila, this RNase III-type protein is termed Dicer. In the second step, the antisense siRNAs produced combine with, and serve as guides for, a different ribonuclease complex called RNA-induced silencing complex (RISC), which cleaves the homologous single-stranded mRNAs. RISC cuts the mRNA approximately in the middle of the region paired with the antisense siRNA, after which the mRNA is further degraded. dsRNAs from different sources can enter the processing pathway leading to RNAi/PTGS.

Thus, in a preferred embodiment of the present invention, the agent for use in the methods of the present invention is a siRNA of Wnt16. siRNA can be used to reduce the expression level of Wnt16. A siRNA of Wnt16 hybridizes to a Wnt16 mRNA and thereby decreases or inhibits production of Wnt16 protein.

In designing RNAi experiments there are several factors that need to be considered such as the nature of the siRNA, the durability of the silencing effect, and the choice of delivery system. To produce an RNAi effect, the siRNA that is introduced into the organism should contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 90% or even 100% identity between the sequence of the siRNA and the gene to be inhibited. Sequences less than about 80% identical to the target gene are substantially less effective. Thus, the greater homology between the siRNA of Wnt16 and the Wnt16 gene whose expression is to be inhibited, the less likely expression of unrelated genes will be affected.

In addition, the size of the siRNA is important. Generally, the present invention relates to siRNA molecules of Wnt16, which are double or single stranded and comprise at least about 19-25 nucleotides, and are able to modulate the gene expression of Wnt19. In the context of the present invention, the siRNA is preferably less than 500, 200, 100, 50 or 25 nucleotides in length. More preferably, the siRNA is from about 19 nucleotides to about 25 nucleotides in length.

In one aspect, the invention generally features an isolated siRNA molecule of at least 19 nucleotides, having at least one strand that is substantially complementary to at least ten but no more than thirty consecutive nucleotides of Wnt16, and that reduces the expression of Wnt16 gene or protein. In a preferred embodiment of the present invention, the siRNA molecule has at least one strand that is substantially complementary to at least ten, preferably at least 19, but no more than thirty consecutive nucleotides of a human Wnt16 gene as shown in SEQ ID NO:25 (GenBank Accession No. NM_{—}057168) or a human Wnt16 gene as shown, e.g., in GenBank Accession Nos. NM_{—}016087, AF152584, AF169963).

In a preferred embodiment of the present invention, a Wnt16 siRNA nucleic acid sequence is 5′-r(AGAUGGAAAGGCACCCACC)d(TT)-3′ (SEQ ID NO:26). In another preferred embodiment of the present invention, a Wnt16 siRNA nucleic acid sequence comprises the sequence 5′-r(AGAUGGAAAGGCACCCACC)d(TT)-3′ (SEQ ID NO:26).

In a preferred embodiment, the siRNA nucleic acid sequence is 5′-r(GGUGGGUGCCUUUCCAUCU)d(TT)-3′ (SEQ ID NO:27). In another preferred embodiment of the present invention, a Wnt16 siRNA nucleic acid sequence comprises the sequence 5′-r(GGUGGGUGCCUUUCCAUCU)d(TT)-3′ (SEQ ID NO:27).

In yet another preferred embodiment of the present invention, a Wnt16 siRNA comprises a duplex formed between 5′-r(AGAUGGAAAGGCACCCACC)d(TT)-3′ (SEQ ID NO:26) and 5′-r(GGUGGGUGCCUUUCCAUCU)d(TT)-3′ (SEQ ID NO:27).

In a preferred embodiment, the siRNA nucleic acid sequence is 5′-r(UGGCAUUGCAACCAGAGAG)d(TT)-3′ (SEQ ID NO:28). In another preferred embodiment of the present invention, a Wnt16 siRNA nucleic acid sequence comprises the sequence 5′-r(UGGCAUUGCAACCAGAGAG)d(TT)-3′ (SEQ ID NO:28).

In a preferred embodiment, the siRNA nucleic acid sequence is 5′-r(CUCUCUGGUUGCAAUGCCA)d(TT)-3′ (SEQ ID NO:29). In another preferred embodiment of the present invention, a Wnt16 siRNA nucleic acid sequence comprises the sequence 5′-r(CUCUCUGGUUGCAAUGCCA)d(TT)-3′ (SEQ ID NO:29).

In yet another preferred embodiment of the present invention, a Wnt16 siRNA comprises a duplex formed between 5′-r(UGGCAUUGCAACCAGAGAG)d(TT)-3′ (SEQ ID NO:28) and 5′-r(CUCUCUGGUUGCAAUGCCA)d(TT)-3′ (SEQ ID NO:29).

In a preferred embodiment, the siRNA nucleic acid sequence is 5′-r(GGAAACUGGAUGUGGUUGG)d(TT)-3′ (SEQ ID NO:30). In another preferred embodiment of the present invention, a Wnt16 siRNA nucleic acid sequence comprises the sequence 5′-r(GGAAACUGGAUGUGGUUGG)d(TT)-3′ (SEQ ID NO:30).

In a preferred embodiment, the siRNA nucleic acid sequence is 5′-r(CCAACCACAUCCAGUUUCC)d(TT)-3′ (SEQ ID NO:31). In another preferred embodiment of the present invention, a Wnt16 siRNA nucleic acid sequence comprises the sequence 5′-r(CCAACCACAUCCAGUUUCC)d(TT)-3′ (SEQ ID NO:31).

In yet another preferred embodiment of the present invention, a Wnt16 siRNA comprises a duplex formed between 5′-r(GGAAACUGGAUGUGGUUGG)d(TT)-3′ (SEQ ID NO:30) and 5′-r(CCAACCACAUCCAGUUUCC)d(TT)-3′ (SEQ ID NO:31).

In a preferred embodiment, the siRNA nucleic acid sequence is 5′-r(UGCAACCGUACAUCAGAGG)d(TT)-3′ (SEQ ID NO:32). In another preferred embodiment of the present invention, a Wnt16 siRNA nucleic acid sequence comprises the sequence 5′-r(UGCAACCGUACAUCAGAGG)d(TT)-3′ (SEQ ID NO:32).

In a preferred embodiment, the siRNA nucleic acid sequence is 5′-r(CCUCUGAUGUACGGUUGCA)d(TT)-3′ (SEQ ID NO:33). In another preferred embodiment of the present invention, a Wnt16 siRNA nucleic acid sequence comprises the sequence 5′-r(CCUCUGAUGUACGGUUGCA)d(TT)-3′ (SEQ ID NO:33).

In yet another preferred embodiment of the present invention, a Wnt16 siRNA comprises a duplex formed between 5′-r(UGCAACCGUACAUCAGAGG)d(TT)-3′ (SEQ ID NO:32) and 5′-r(CCUCUGAUGUACGGUUGCA)d(TT)-3′ (SEQ ID NO:33).

In another preferred embodiment, the siRNA molecule of Wnt2 includes a sequence that is at least 90% homologous, preferably 95%, 99%, or 100% homologous, to the nucleic acid sequences shown in SEQ ID NOS:25, 26, 27, 28, 29, 30, 31, or 32. Without undue experimentation and using the disclosure of this invention, it is understood that additional siRNAs of Wnt16 that modulate Wnt16 expression can be designed and used to practice the methods of the invention.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In a preferred embodiment, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in the published U.S. application publication number 20040019001 and U.S. Pat. No. 6,673,611 (incorporated by reference). Collectively, all such altered RNAs described above are referred to as modified siRNAs.

Preferably, RNAi is capable of decreasing the expression of Wnt16 in a cell by at least 10%, 20%, 30%, or 40%, more preferably by at least 50%, 60%, or 70%, and most preferably by at least 75%, 80%, 90%, 95% or more.

Introduction of siRNA into cells can be achieved by methods known in the art, including for example, microinjection, electroporation, or transfection of a vector comprising a nucleic acid from which the siRNA can be transcribed. Alternatively, a siRNA for Wnt16 can be directly introduced into a cell in a form that is capable of binding to Wnt16 mRNA transcripts. To increase durability and membrane-permeability the siRNA may be combined or modified with liposomes, poly-L-lysine, lipids, cholesterol, lipofectine or derivatives thereof. Preferred are cholesterol-conjugated siRNA for Wnt16 (see, Song et al., Nature Med. 9:347-351 (2003)).

F. Anti-Wnt16 Antibodies for Use in the Methods of the Invention

In another preferred embodiment of the present invention, the agent used in the methods of the present invention is an anti-Wnt16 antibody as fully described herein. The anti-Wnt16 antibody can be a polyclonal, a monoclonal anti-Wnt16 antibody or an anti-Wnt16 Fab as fully described herein. Preferably, the methods of the present invention use an anti-Wnt16 monoclonal antibody. Also preferred are anti-Wnt16 Fab. Particularly preferred is a human anti-Wnt16 monoclonal antibody or a human anti-Wnt16 Fab.

### IV. Identification of Inhibitors of Wnt Signaling

Wnt16 protein (or cells expressing them) or members of the Wnt signaling pathway, e.g., dvl, can also be used in drug screening assays to identify agents that inhibit Wnt signaling. The present invention thus provides novel methods for screening for compositions which inhibit cancer.

Assays for Wnt16 signaling can be designed to detect and/or quantify any part of the Wnt16 signaling pathway. For example the ability of an agent to affect intracellular α-catenin levels or to induce apoptosis in target cells can be measured. Assays suitable for these purposes are described herein.

Assays may include those designed to test binding activity of an inhibitor to either the Wnt16 ligand, the Frizzled receptor, or another member of the Wnt16 signaling cascade, e.g., dvl. These assays are particularly useful in identifying agents that modulate Wnt16 activity. Virtually any agent can be tested in such an assay. Such agents include, but are not limited to natural or synthetic polypeptides, antibodies, natural or synthetic small organic molecules, nucleic acids and the like.

As noted above, a family of secreted Frizzled-related proteins (sFRPs) function as soluble endogenous modulators of Wnt signaling by competing with Frizzled receptors for the binding of secreted Wnt ligands. Thus, in some format, test agents are based on natural ligands (e.g., Wnt ligands or sFRPs) of the Frizzled receptor.

Any of the assays for detecting Wnt16 signaling are amenable to high throughput screening. High throughput assays binding assays and reporter gene assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

Other assays useful in the present invention are those designed to test neoplastic phenotypes of cancer cells. These assays include cell growth on soft agar; anchorage dependence; contact inhibition and density limitation of growth; cellular proliferation; cell death (apoptosis); cellular transformation; growth factor or serum dependence; tumor specific marker levels; invasiveness into Matrigel; tumor growth and metastasis in vivo; mRNA and protein expression in cells undergoing metastasis, and other characteristics of cancer cells.

The ability of test agents to inhibit cell growth can also be assessed by introducing the test into an animal model of disease, and assessing the growth of cancer cells in vivo. For example, human tumor cells can be introduced into an immunocompromised animal such as a “nude mouse”. The test agent (e.g., a small molecule or an antibody) is administered to the animal and the ability of the tumor cell to form tumors—as assessed by the number and/or size of tumors formed in the animal—is compared to tumor growth in a control animal without the agent.

A. Inhibitors of Gene Expression

In one aspect of the present invention, inhibitors of the Wnt16 signaling pathway, e.g., Dvl inhibitors, can comprise nucleic acid molecules that inhibit expression of the target protein in the pathway. Conventional viral and non-viral based gene transfer methods can be used to introduce nucleic acids encoding engineered polypeptides, e.g., dominant negative forms of the protein, in mammalian cells or target tissues, or alternatively, nucleic acids e.g., inhibitors of target protein expression, such as siRNAs or anti-sense RNAs. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1:13-26 (1994).

In some embodiments, small interfering RNAs are administered. In mammalian cells, introduction of long dsRNA (>30 nt) often initiates a potent antiviral response, exemplified by nonspecific inhibition of protein synthesis and RNA degradation. The phenomenon of RNA interference is described and discussed, e.g., in Bass, Nature 411:428-29 (2001); Elbahir et al., Nature 411:494-98 (2001); and Fire et al., Nature 391:806-11 (1998), where methods of making interfering RNA also are discussed. The siRNA inhibitors are less than 100 base pairs, typically 30 bps or shorter, and are made by approaches known in the art. Exemplary siRNAs according to the invention can have up to 29 nucleotides, 25 nucleotides, 22 nucleotides, 21 nucleotides, 20 nucleotides, 15 nucleotides, 10 nucleotides, 5 nucleotides or any integer thereabout or therebetween.

### V. Pharmaceutical Compositions

As noted above, inhibitors of Wnt16 expression and agents of the present invention can be used to treat a disease associated with Wnt16 signaling, such as a cancer associated with Wnt16 signaling. The compositions for administration will commonly comprise an agent, as fully described herein, dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patients needs.

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that antibodies when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions containing inhibitors and/or agents of the invention (e.g., antibodies) can be administered for therapeutic or prophylactic treatments. In therapeutic applications, compositions are administered to a patient suffering from a disease (e.g., breast cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of an inhibitor that is capable of preventing or slowing the development of cancer in a patient is referred to as a “prophylactically effective dose.” The particular dose required for a prophylactic treatment will depend upon the medical condition and history of the patient, the particular cancer being prevented, as well as other factors such as age, weight, gender, administration route, efficiency, etc. Such prophylactic treatments may be used, e.g., in a patient who has previously had cancer to prevent a recurrence of the cancer, or in a patient who is suspected of having a significant likelihood of developing cancer.

A “patient” or “subject” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

Other known cancer therapies can be used in combination with the methods of the invention. For example, inhibitors of Wnt16 signaling may also be used to target or sensitize a cell to other cancer therapeutic agents such as 5FU, vinblastine, actinomycin D, cisplatin, methotrexate, and the like. In other embodiments, the methods of the invention can be used with radiation therapy and the like.

In some instances an antibody belongs to a sub-type that activates serum complement when complexed with the transmembrane protein thereby mediating cytotoxicity or antigen-dependent cytotoxicity (ADCC). Thus, cancer can be treated by administering to a patient antibodies directed against Frizzled proteins on the surface of cancer cells. Antibody-labeling may activate a co-toxin, localize a toxin payload, or otherwise provide means to locally ablate cells. In these embodiments, the antibody is conjugated to an effector moiety. The effector moiety can be any number of molecules, including labeling moieties such as radioactive labels or fluorescent labels, or can be a therapeutic moiety, such as a cytotoxic agent.

A. Use of Wnt16 Polypeptides as Vaccines

In addition to administration of inhibitors of Wnt16 signaling, the Wnt16 proteins or immunogenic fragments of them can be administered as vaccine compositions to stimulate HTL, CTL, and antibody responses against the endogenous proteins. Such vaccine compositions can include, e.g., lipidated peptides (see, e.g., Vitiello, et al. (1995) J Clin Invest. 95:341-349), peptide compositions encapsulated in poly(D,L-lactide-co-glycolide, “PLG”) microspheres (see, e.g. Eldridge et al. (1991) Molec Immuno 28:287-294; Alonso et al. (1994) Vaccine 12:299-306; Jones et al. (1995) Vaccine 13:675-681), peptide compositions contained in immune stimulating complexes (ISCOMS; see, e.g., Takahashi et al. (1990) Nature 344:873-875; Hu et al. (1998) Clin Exp Immunol 113:235-243), multiple antigen peptide systems (MAPs; see, e.g., Tam (1988) Proc Natl Acad Sci USA 85:5409-5413; Tam (1996) J Immunol Methods 196:17-32); viral delivery vectors (Perkus et al., p. 379, in Kaufmann (ed. 1996) Concepts in Vaccine Development de Gruyter; Chakrabarti et al. (1986) Nature 320:535-537; Hu et al. (1986) Nature 320:537-540; Kieny et al. (1986) AIDS Bio/Technology 4:790-795; Top et al. (1971) J Infect Dis 124:148-154; Chanda et al. (1990) Virology 175:535-547), particles of viral or synthetic origin (see, e.g., Kofler et al. (1996) J Immunol Methods 192:25-35; Eldridge et al. (1993) Sem Hematol 30:16-24; Falo et al. (1995) Nature Med 7:649-653).

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis, or Mycobacterium tuberculosis derived proteins. Certain adjuvants are commercially available as, e.g., Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Vaccines can be administered as nucleic acid compositions wherein DNA or RNA encoding the Wnt16 polypeptides, or a fragment thereof, is administered to a patient. See, e.g., Wolff et al. (1990) Science 247:1465-1468; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

Methods for the use of genes as DNA vaccines are well known, and include placing the desired gene or portion thereof under the control of a regulatable promoter or a tissue-specific promoter for expression in the patient. The gene used for DNA vaccines can encode full-length Wnt16 protein, or may encode portions of the proteins.

In a some embodiments, the DNA vaccines include a gene encoding an adjuvant molecule with the DNA vaccine. Such adjuvant molecules include cytokines that increase the immunogenic response to the polypeptide encoded by the DNA vaccine.

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, e.g., as a vector to express nucleotide sequences that encode Wnt or Frizzled polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover, et al. (1991) Nature 351:456-460. A wide variety of other vectors useful for therapeutic administration or immunization e.g., adeno and adeno-associated virus vectors, retroviral vectors, Salmonella typhi vectors, detoxified anthrax toxin vectors, and the like, will be apparent. See, e.g., Shata et al. (2000) Mol Med Today 6:66-71; Shedlock et al. (2000) J Leukoc Biol 68:793-806; and Hipp et al. (2000) In Vivo 14:571-85.

### VI. Administration of Inhibitors and Agents

The agents that inhibit Wnt16 signaling (e.g., anti-Wnt16 antibodies and siRNA) can be used in a variety of therapeutic regimens. For example, the agents can be used in methods comprising, but not limited to parenteral (e.g., intravenous, intramuscular, intradermal, intraperitoneal, and subcutaneous routes), topical, oral, local, or transdermal administration. These methods can be used for prophylactic and/or therapeutic treatment.

A. Non-Viral Delivery Methods

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther 2:291-297 (1995); Behr et al., Bioconjugate Chem. 5:382-389 (1994); Remy et al., Bioconjugate Chem 5:647-654 (1994); Gao et al., Gene Therapy 2:710-722 (1995); Ahmad et al., Cancer Res 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

B. Viral Delivery Methods

The use of RNA or DNA viral based systems for the delivery of inhibitors of target Wnt pathway proteins, e.g., Dvl, are known in the art. Conventional viral based systems for the delivery of such nucleic acid inhibitors can include retroviral, lentivirus, adenoviral, adeno-associated and herpes simplex virus vectors for gene transfer.

In many gene therapy applications, it is desirable that the gene therapy vector be delivered with a high degree of specificity to a particular tissue type, e.g., a lung cancer. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., Proc Natl Acad Sci USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient.

Ex vivo cell transfection for diagnostics, research, or for gene therapy (e.g., via re-infusion of the transfected cells into the host organism) is well known to those of skill in the art. In some embodiments, cells are isolated from the subject organism, transfected with inhibitor nucleic acids and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Vectors (e.g., retroviruses, adenoviruses, liposomes, etc.) containing therapeutic nucleic acids can also be administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention, as described below (see, e.g., Remington's Pharmaceutical Sciences, 17th ed., 1989).

### VII. Kits for Use in Diagnostic, Research, and Therapeutic Applications

The invention also provides kits that can be used for the detection of the Wnt16 nucleic acids or proteins disclosed herein. Further, kits are provided comprising compositions described herein that allow the user to practice the methods of the invention. In diagnostic and research applications such kits may include any or all of the following: assay reagents, buffers, Wnt16-specific or Frizzled-specific nucleic acids or monoclonal or polyclonal antibodies, hybridization probes and/or primers, and the like. A therapeutic product may include sterile saline or another pharmaceutically acceptable emulsion and suspension base.

In a preferred embodiment, the kit comprises an agent embracing the specifics as outlined herein, wherein the agent binds Wnt16 protein or a Wnt16 nucleic acid, such as mRNA, interferes with Wnt16 signaling, or inhibits binding of Wnt16 protein to other proteins, such as a Frizzled receptor. The kit may further comprise one or more containers for agents and compositions of the present invention and instructions for using the agent to inhibit the proliferation of a cell overexpressing Wnt16, to treat a disease, such as a cancer overexpressing Wnt16, to induce apoptosis in a cell overexpressing Wnt16, to detect a cancer cell overexpressing Wnt16 or to practice any of the methods described herein.

In a preferred embodiment of the invention, a kit comprises a siRNA as shown in SEQ ID NO: 26, 27, 28, 29, 30, 31, 32 or 33. In another preferred embodiment a kit comprises a siRNA comprising a nucleic acid sequence as shown in SEQ ID NO: 26, 27, 28, 29, 30, 31, 32 or 33. In yet another preferred embodiment of the present invention, the kit comprises or a siRNA of about 19-25 contiguous nucleotides of SEQ ID NO:25, wherein the siRNA binds to Wnt16 mRNA and inhibits translation of Wnt16 mRNA. Optionally, this kit further comprises one or more containers for agents and compositions of the present invention and instructions for using the siRNA to inhibit the proliferation of a cell overexpressing Wnt16, to treat a disease, such as a cancer overexpressing Wnt16, to induce apoptosis in a cell overexpressing Wnt16, to detect a cancer cell overexpressing Wnt16 or to practice any of the methods described herein. The kit may further comprise a control or non-silencing siRNA.

As indicated above, the kits of the invention may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides kits for screening for inhibitors of Wnt16 signaling. Such kits can be prepared from readily available materials and reagents. For example, such kits comprise one or more of the following materials: a Wnt16 polypeptide or polynucleotide, reaction tubes and instructions for testing the desired Wnt16 signaling function (e.g., β-catenin levels).

Pharmaceutical compositions and kits of the present invention embrace the specifics as outlined herein.

Although the forgoing invention has been described in some detail by way of illustration and example for clarity and understanding, it will be readily apparent to one ordinary skill in the art in light of the teachings of this invention that certain variations, changes, modifications and substitution of equivalents may be made thereto without necessarily departing from the spirit and scope of this invention. As a result, the embodiments described herein are subject to various modifications, changes and the like, with the scope of this invention being determined solely by reference to the claims appended hereto.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The invention is further illustrated by the following examples, which are only illustrative and are not intended to limit the definition and scope of the invention in any way.

### VIII. EXAMPLES

### Example 1

### Materials and Methods

1. Cell Lines

Four Human pre-B ALL cell lines were studied. CCL-119 (CCRF-CEM) was obtained from American Type Culture Collection (ATCC, Manassas, Va., USA), NALM-6 and 697 (ACC-42) were obtained from German Collections of Microorganisms and Cell Cultures (DSMZ, Braunschweig, Germany), and RCH-ACV was kindly provided by Dr. Mignon Loh (Department of Pediatrics, University of California San Francisco, Calif., USA). Both 697 and RCH-ACV cell lines (acute lymphoblastoid leukemia) display a t(1;19) chromosomal translocation and have considerable E2a-Pbx1 expression whereas NALM-6 and CCL-119 (acute lymphoblastoid leukemia) do not and therefore served as a control. Human non-small-cell lung cancer (NSCLC) cell line H460 and breast cancer cell line MCF7 were obtained from American Type Culture Collection. All cells were grown in RPMI 1640 supplemented with 10% fetal bovine serum, penicillin (100 IU/ml) and streptomycin (100 mg/ml). All cells were cultured at 37° C. in a humid incubator with 5% CO₂.

2. RT-PCR

Expression analysis of Wnt16 in cell lines was carried out by RT-PCR. Total RNA from all cell lines was isolated using an extraction kit (RNeasy Mini Kit, Qiagen, Valencia, Calif., USA). RNA quantification was determined by measuring the absorbance at 260 nm in a spectrophotometer. RT-PCR was performed in GeneAmp PCR system 9700 using a kit (SuperScript One-Step RT-PCR with Platinum Taq, Invitrogen, Carlsbad, Calif., USA). Primer sequences used for Wnt16a and Wnt16b were the following: Wnt16aF: CAGAAAGATGGAAAGGCACC (SEQ ID NO:17), Wnt16bF: TGCTCGTGCTGTTCCCCTAC (SEQ ID NO:18), Wnt16aR and Wnt16bR: ATCATGCAGTTCCATCTCTC (SEQ ID NO: 19) as reported in Fear et al., Biochem. Biophys Res Comm 278:814-820 (2000). In all experiments, 0.2 mM of each primer and 1 μg of each template RNA was used with the RT/platinum Taq mix. PCR conditions were 1 cycle at 50° C. for 30 minutes and at 94° C. for 2 minutes then 35 cycles at 94° C. for 15 seconds, at 57° C. for 30 seconds and at 72° C. for 1 minute followed by 1 cycle at 72° C. for 7 minutes.

3. Western Blotting

Standard protocol was used. Anti-Dvl3, and anti-Survivin antibodies were from Santa Cruz Biotechnology (Santa Cruz, Calif.). Anti-caspase3 antibody was from Oncogene (Cambridge, Mass.). Anti-β-Actin antibody was obtained from Cell Signaling Technology, Inc. (Beverly, Mass.), or Santa Cruz Biotechnology (Santa Cruz, Calif.). Anti-β-Catenin antibody was from Transduction Laboratories (Lexington, Ky.) or (Pharminigen). Anti-Cytochrome c antibody was from BD Biosciences (San Diego, Calif.). For detecting alteration of α-catenin and cytochrome-c, cytosolic extracts were prepared and examined by methods known in the art (e.g., Wang et al., Mol Cell Biol 19(9):5923-9 (1999)).

4. RNA Interference

The above ALL cell lines were plated into 6-well plates with fresh media without antibiotics 24 hours before experiments. One non-silencing siRNA purchased from Ambion, (Austin, Tex., USA) was used as a control, along with one siRNA targeting Wnt16a and three siRNA targeting specifically Wnt16b purchased from Qiagen (Valencia, Calif., USA) were used. The sequences for the silencing siRNA were the following:

(SEQ ID NO:26)

| | |
|---|---|
| Wnt16a: | 5′-r(AGAUGGAAAGGCACCCACC)d(TT)-3′ |

(SEQ ID NO:27)

| | |
|---|---|
| Wnt16a: | 5′-r(GGUGGGUGCCUUUCCAUCU)d(TT)-3′ |

(SEQ ID NO:28)

| | |
|---|---|
| Wnt16b1: | 5′-r(UGGCAUUGCAACCAGAGAG)d(TT)-3′ |

(SEQ ID NO:29)

| | |
|---|---|
| Wnt16b1: | 5′-r(CUCUCUGGUUGCAAUGCCA)d(TT)-3′ |

(SEQ ID NO:30)

| | |
|---|---|
| Wnt16b2: | 5′-r(GGAAACUGGAUGUGGUUGG)d(TT)-3′ |

(SEQ ID NO:31)

| | |
|---|---|
| Wnt16b2: | 5′-r(CCAACCACAUCCAGUUUCC)d(TT)-3′ |

(SEQ ID NO:32)

| | |
|---|---|
| Wnt16b3: | 5′-r(UGCAACCGUACAUCAGAGG)d(TT)-3′ |

(SEQ ID NO:33)

| | |
|---|---|
| Wnt16b3: | 5′-r(CCUCUGAUGUACGGUUGCA)d(TT)-3′ |

We followed the protocol proposed by Elbashir et al., Methods 26:199-213 (2002). Transfections were done with Oligofectamine Reagent (Invitrogen Life Technologies, Carlsbad, Calif.). After siRNA transfection, plates were incubated from 72 to 96 hours at 37° C. before further analysis.

5. Apoptosis Analysis

The ability of the selected siRNA to induce apoptosis was evaluated in leukemia cell lines, lung cancer cell lines and other cell lines. In brief, 72 hours after transfection, cells were harvested by trypsinization and processed for determination of cell surface annexin-V and propidium iodide (PI) contents (Apotarget, BioSource International, Camarillo, Calif., USA) according to the manufacturer's protocol. With the use of Annexin-V-PI double staining regime, three populations of cells are distinguishable in two color flow cytometry: (a) non-apoptotic cells: annexin-V and PI negative; (b) early apoptotic cells with exposed phosphatidylserine but intact cell membranes bound to Annexin V-FITC but excluded propidium iodide; and (c) cells in necrotic or late apoptotic stages were labeled with both Annexin-VFITC and PI. Then stained cells were immediately analyzed by flow cytometry (FACScan, Decton, Dickinson). For each experiment, compensation was done by using unstained, Annexin-V only stained and PI only stained samples.

6. Anti-Wnt16 Antibodies

Mouse anti-human Wnt16 polyclonal antibody was purchased from Santa Cruz Biotechnology (Santa Cruz, Calif., USA) and used for Western-Blotting. Another mouse anti-Wnt16 monoclonal antibody was purchased from BD Pharmingen (cat#552595).

Human Fab antibodies were custom-made using as an antigen a peptide comprising amino acid residues 1-99 of the N-terminal region of human Wnt16 protein as shown in SEQ ID NO:2. Amino acid residues 1-99 (including the signal peptide sequence) of Wnt16 were expressed in E. coli using standard protocols. The purified peptide was then used as an antigen. Fabs were made by HuCal®-EST technology by Antibody by Design (MorphoSys, Germany). Eight human Fabs were generated and named 582, 583, 584, 585, 586, 587, 588, and 589. Human anti-Wnt16 Fabs were used for the inhibition experiments described herein.

7. ELISA for Determining Specificity of Human Fabs.

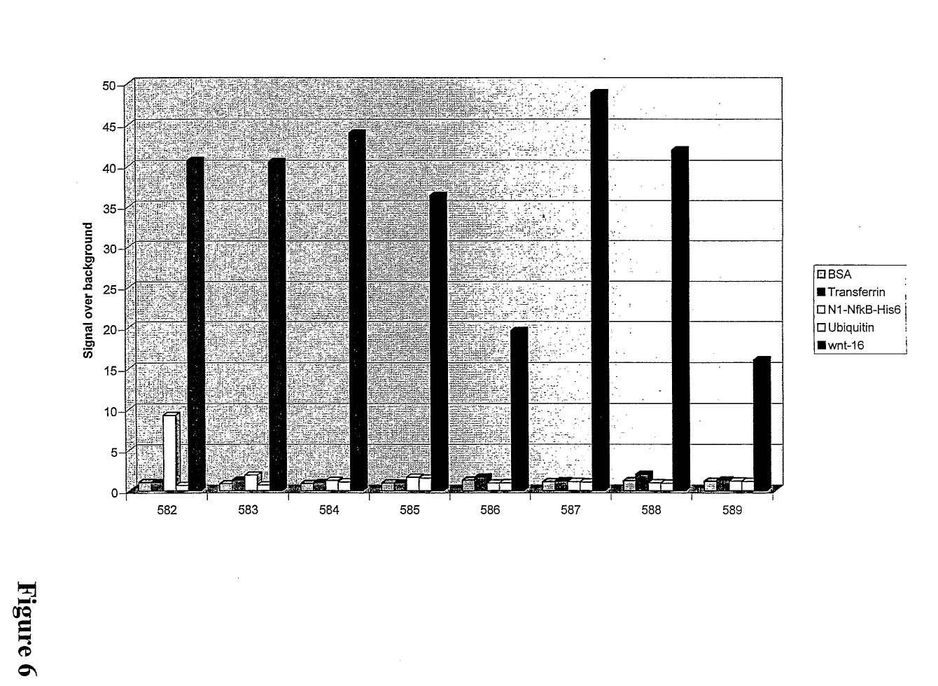

Direct ELISA was used to determine the specificity of the human anti-Wnt16 Fabs. Briefly, BSA, transferring, N1-NfkB-His6, Ubiquitin and the Wnt16 antigen (amino acids 1-99 of unprocessed human Wnt16 as shown in SEQ ID NO:2) at a concentration of 10 μg/ml in PBS were used to coat plates for 12 hours at 4° C. The plates were then incubated in blocking buffer for 2 hours at room temperature before the human Fabs were added. A representative example of eight anti-Wnt16 Fabs (clones 582-589) that tested positive in this assay is shown in FIG. 6.

8. Cell Surface Antigen Binding Analysis

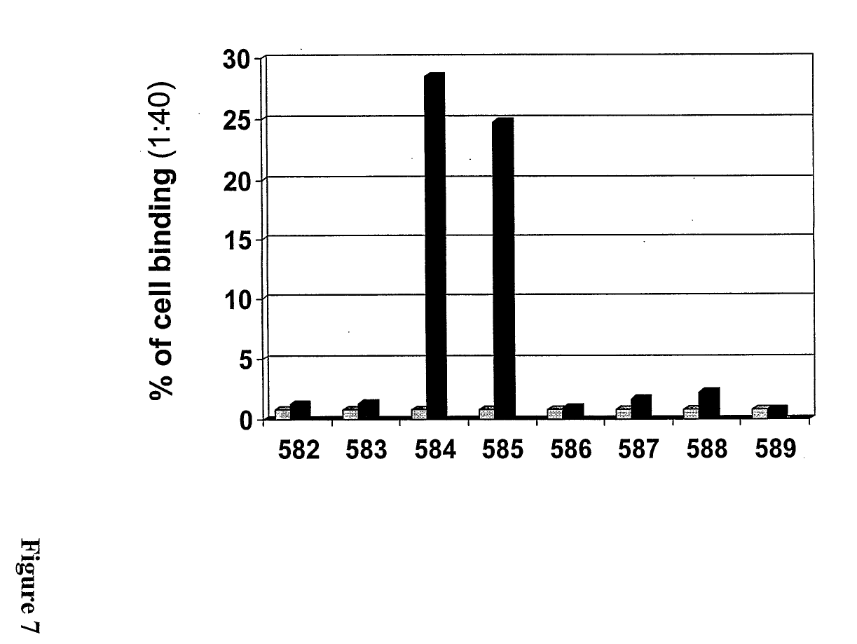

Cells to be tested were resuspended in 100 μl PBS buffer containing 0.5-1 μg of human anti-Wnt16 Fab and incubated on ice for 30 min. After several washing steps, the cells were resuspended in 100 μl FACS buffer containing 1 μg secondary antibody, vortexed and incubated on ice for 30 min. Incubation was performed in the dark. The secondary antibody used for FACS was Alexa Flour 488 goat anti-human IgG (H+L)₆. The samples were then placed in 12×75 mm Falcon tubes and analyzed by flow cytometry. A representative example for human anti-Wnt16 Fab binding to the surface of RCH-ACV cells is shown in FIG. 7.

9. Gene Expression Array

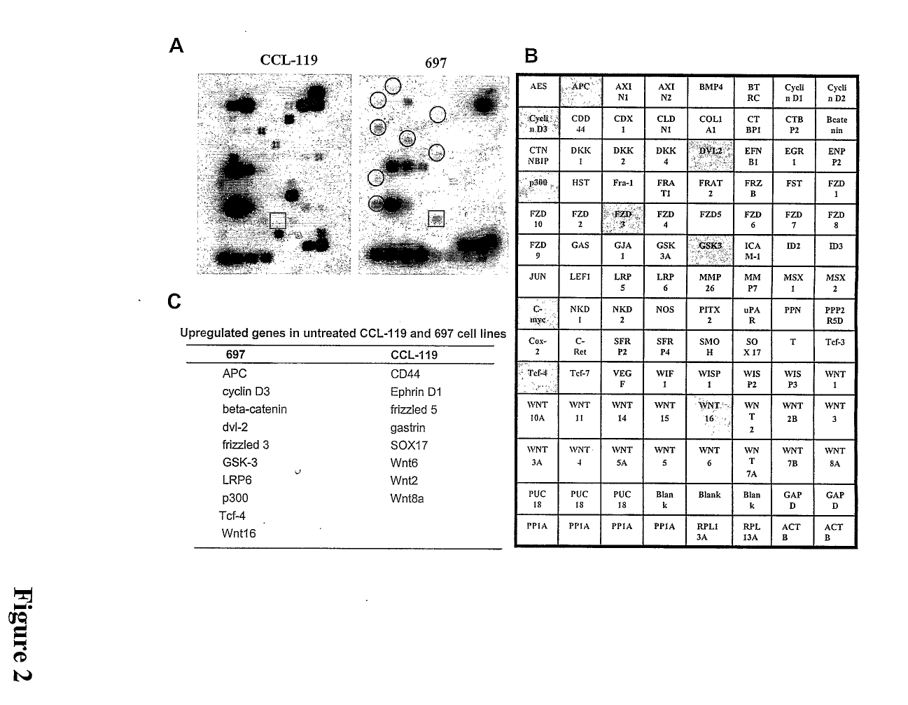

In some experiments, gene expression profiling was analyzed using a custom array designed to profile the expression of genes involved in and downstream of Wnt signaling with the AmpoLabelling-LPR Kit protocol (GEArray Q Human Wnt Signaling Pathway Gene Array, SuperArray, Frederick, Md., USA). Briefly, total RNA isolated from the selected cell lines was subjected to an RT reaction and cDNA probes were subsequently labeled with Biotin-16-dUTP (Roche, Basel, Switzerland), denatured and hybridized overnight in hybridization tubes containing the Wnt specific arrays. Using a CDD camera, detection was done with a chemiluminescent reaction and the membranes were exposed to X-ray film with multiple exposures for various times. Images of spots were converted in numerical data using software provided by the manufacturer. Expression data was matched against the gene list provided by the manufacturer. A representative gene expression profile is shown in FIG. 2.

10. Proliferation Assay

Alimta® (MTA), supplied by Eli Lilly (Indianapolis, Ind.) is diluted in sterile physiological solution at a concentration of 10 mg/ml. The stock is divided into aliquots, stored at −80° C., and diluted in culture medium before each experiment. Anti-Wnt16 antibody is used as previously described. Cell proliferation can be determined by measuring metabolic activity of tetrazolium conversion (Cell Titer 96 assay, Promega, Madison, Wis.). Briefly, 5,000 cells are plated per well in a 96-well plate and culture medium containing increasing concentrations of both drugs is added. Plates are incubated at 37° C. for 72 hours in a CO₂ incubator. Then a solubilization/stop solution is added and the absorbance is recorded with a fluorescence plate reader at a wavelength of 570 nm. Each experiment is repeated at least 3 times in triplicate.

11. In Vivo Tumor Suppression Studies

Female nude mice, 5-6 weeks old, may be injected s.c. with about 1×10⁷ ALL cells, pre-B ALL cells, CLL cells or H460 cells in the dorsal area in a volume of about 100 μl. When tumors have uniformly formed the animals can then be intraperitoneally (i.p.) injected with a monoclonal anti-Wnt16 antibody, a control monoclonal antibody, or PBS buffer in a volume of about 100 μl. A preferred dose of the monoclonal anti-Wnt16 and control antibodies for injection is about 250 μg. Each injection may be done twice weekly. Preferably, each group consists of at least 5-10 mice. Tumor size may then be determined at weekly intervals, and tumor volumes may be calculated using width (x) and length (y) (x²y/2, where x<y) (Sonoda et al., Cancer Res 61(13):4956-60 (2001)).

12. Statistical Analysis

Data shown represent mean values (±S.E.M.). Unpaired T-Test in the Excel was used for comparing different treatments and cell lines. Other statistical comparisons were made with a two-sided Student's t-test (P<0.01).

### Example 2

### Wnt16 and Wnt Signaling Pathway are Upregulated in Leukemia Cell Lines Containing the t(1;19) Translocation

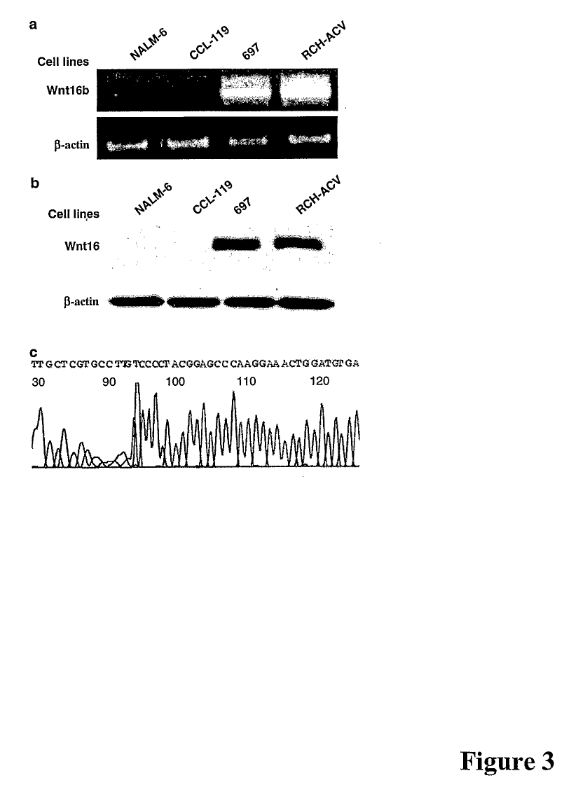

In order to analyze the Wnt signaling pathway activation, gene expression profiling using a custom array designed to profile the expression of the Wnt signaling pathway-related genes was performed. Four acute lymphoblastoid leukemia cell lines were analyzed, two containing the t(1;19) translocation (697 and RCH-ACV) and two without the translocation (CCL-119 and NALM-6). Wnt16 over expression in both 697 and RCH-ACV cell lines was confirmed whereas no signal was seen in the control cell lines CCL-119 and NALM-6. Moreover, many other genes involved in the Wnt canonical pathway such as adenomatous polyposis coli (APC), β-catenin, dishevelled (dvl)-2 and Tcf-4 were over expressed, underlining the activation of the Wnt signaling pathway in those cells (FIG. 3A). It is notable that other Wnt proteins (Wnt2 and Wnt6) are over expressed in CCL-119, suggesting that various Wnt proteins may be upregulated in different leukemia subtypes in agreement with the findings of Muller-Tidow et al. (Mol. Cell. Biol. 24:2890-28904 (2004)).

### Example 3

### Wnt16a and Wnt16b Expression in Leukemia Cell Lines

To further confirm the previous data, Wnt16 expression was further analyzed by both Western-Blot and RT-PCR. First, by using a commercially available Wnt16 polyclonal antibody (Santa Cruz Biotechnologies), it was demonstrated that Wnt16 is not expressed in both NALM-6 and CCL-119 control cell lines and is highly overexpressed in both 697 and RCH-ACV (FIG. 3B). Moreover, as Fear et al. (supra) showed that Wnt16 consists in two isoforms, we analyzed by RT-PCR the expression of both Wnt16a and Wnt16b in the same cell lines. RT-PCR was performed with the same primers as those used by Fear et al. (supra) and showed a marked overexpression of Wnt16b in t(1;19)-containing cell lines (FIG. 3A) compared to the others, whereas Wnt16a was not detected at all in the four cell lines. Moreover, as Fear et al. (supra) suggested that Wnt16a was more likely to be targeted by E2A-Pbx1 than Wnt16b, it was confirmed by sequencing analysis that the amplified band corresponded strictly to Wnt16b (FIG. 3C).

### Example 4

### Wnt16b Inhibition by siRNA Leads to Apoptosis

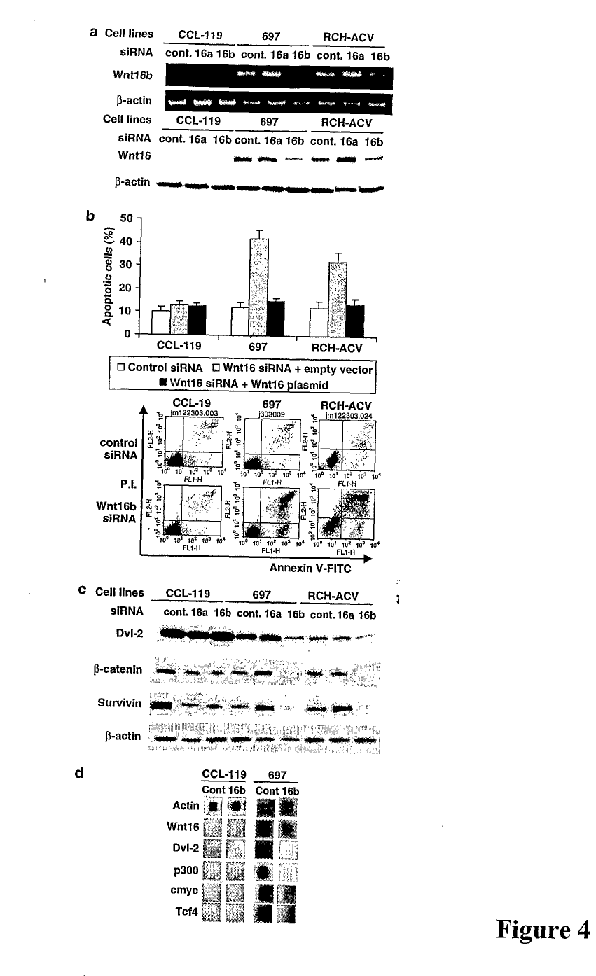

As Wnt16b appeared to be a putative target to mediate the effects of the fusion protein E2A-Pbx1, its ability to control apoptosis and cell survival was studied. RNA interference was carried out by following the protocol described by Elbashir et al. (Methods 26(2):199-213 (2002)). Specific siRNA for Wnt16a and Wnt16b were designed. Four siRNA were used, one nonsilencing siRNA, one siRNA targeting Wnt16a (see, SEQ ID NOS: 26 and 27) and three siRNA targeting specifically Wnt16b (see, SEQ ID NOS: 28 and 29; SEQ ID NOS: 30 and 31; and SEQ ID NOS: 32 and 33). ALL cell lines were transfected by the above siRNA and were incubated from 72 to 96 h before further analysis. It was first demonstrated by both RT-PCR and Western blotting that Wnt16b-specific siRNA inhibited Wnt16b, whereas Wnt16a siRNA and control siRNA had no effect (FIG. 4A). Then apoptosis was assessed. Flow cytometry analysis after annexin-V and propidium iodide treatment revealed that treatment with Wnt16b siRNA for 3-5 days induced apoptosis and cell death in pre-B ALL cell lines expressing Wnt16b, whereas controls did not. Three different Wnt16 siRNAs were used giving similar results in various experiments. Significant apoptosis (typically at least 35% to 40%) was induced by 100 nM Wnt16b siRNA (FIG. 4B), and no apoptosis was induced by Wnt16a siRNA or non-silencing siRNA control (100 nM) (P<0.01). The silencing of Wnt16 expression after Wnt16b siRNA treatments (100 nM for 72 hrs) was confirmed by Western blot analysis. Non-silencing siRNA served as control (100 nM for 72 hrs).

### Example 5

### Coexpression of Wnt16b Rescues Cells From siRNA-Induced Apoptosis

In order to test whether coexpression of Wnt16b cDNA can rescue the cells from the siRNA-induced apoptosis we transfected a Wnt16 pcDNA3 plasmid (obtained from Dr. YingziYangat the NHGRI/NIH) into the cells. The Wnt16 siRNAs described herein can not bind to this plasmid or mRNA transcribed therefrom, because the Wnt16 siRNA were designed to bind to the 3′ UTR region of Wnt16 mRNA, which is not present in the Wnt16 pcDNA3 plasmid. Thus, the siRNA can only silence expression of the endogenous Wnt16. It was demonstrated that re-expression of Wnt16 can rescue the cells from the siRNA-induced apoptosis (FIG. 4B).

### Example 6

### Expression of Wnt16 Downstream Effectors after Silencing of Wnt16

In the canonical Wnt pathway, Wnt proteins bind to frizzled receptors, activate dishevelled proteins which inhibits the ability of glycogen synthase kinase-3 P to phosphorylate β-catenin that can in turn enter the cell nucleus, and activate target genes. The expression of the Wnt pathway's downstream effectors was thus analyzed. Western Blot analysis performed on the cell lines showed that Wnt16b inhibition led to a decrease in β-catenin, Dvl-2 and survivin expression in t(1;19)-containing cell lines, whereas no effect was observed on other cell lines (FIG. 4C). To further confirm these findings, a Wnt signaling specific microarray as previously described was performed in CCL-119 and 697 cell lines treated with either control (nonsilencing) siRNA or with Wnt16b siRNA. First the specific inhibition of Wnt16 was confirmed. Further, it was shown that this inhibition led also to the downregulation of key components of the canonical pathway such as APC, GSK-3, Dvl-2, transcription factors such as Tcf-4 and p300 and other genes such as c-myc and cyclin D3 (FIG. 4D).

Thus, to determine whether the apoptotic effects correlated with the inhibition of Wnt16b signaling, it was shown that expression levels of Dvl-3, cytosolic β-catenin, and Survivin were down-regulated after Wnt16b siRNA treatment. The β-catenin-TCF targeted genes, c-Myc and fibronectin, were also found down-regulated in siRNA Wnt16b treated pre-B ALL cells.

### Example 7

### Development of Monoclonal Antibodies Against Human Wnt16b

Anti-Wnt16 antibodies were raised against polypeptides from human Wnt16b as shown in the sequence listing and as further described herein. Anti-Wnt16b antibodies raised against a full-length Wnt16 as shown in SEQ ID NO:2 and against a peptide comprising amino acid residues 1-99 as shown in SEQ ID NO:2. human anti Wnt16 antibodies Fab1 (clone#582), Fab2 (clone #583), Fab3 (clone#584), Fab4 (clone #585), Fab5 (clone #586), Fab6 (clone #587), Fab7 (clone#588) and Fab8 (clone #599) were obtained. These Anti-Wnt16 antibodies were then tested for specificity, biological activity, the ability to induce apoptosis in the cell lines that have Wnt16b expression. The binding of the anti-Wnt16b antibody to Wnt16b-expressing cells was confirmed by western blotting and flow cytometry.

### Example 8

### Wnt16 Inhibition by Anti-Wnt16 Antibodies Induces Apoptosis in Leukemia Cells

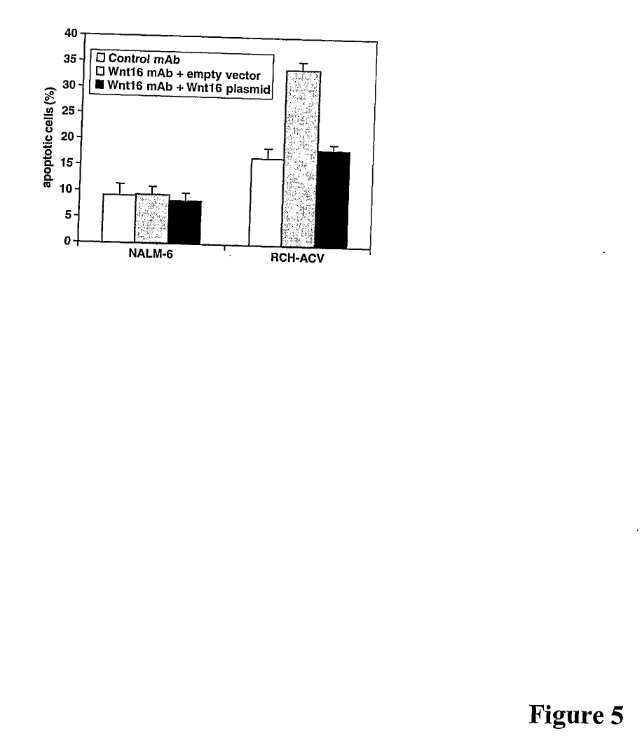

An anti-Wnt16 monoclonal antibody (BD Pharmingen; cat#552595) was used to perform apoptotic assays with the 697 and RCH-ACV cell lines and the control cell line, NALM-6. The leukemia cell lines were stained with Annexin V-FITC and propidium iodide (PI) after antibody treatment. As sown in FIG. 5, the anti-Wnt16 antibody induced apoptosis in the RCH-ACV cell line, whereas no effect was noticed in the NALM-6 cell line. Similarly, apoptosis was induced in the 697 cell line. Further, it was analyzed if re-expression of Wnt16b can rescue the RCH-ACV cells from the anti-Wnt16 antibody-induced apoptosis (see, Example 5 for details). Re-expression of Wnt16 can rescue the cells from the monoclonal antibody-induced apoptosis (FIG. 5).

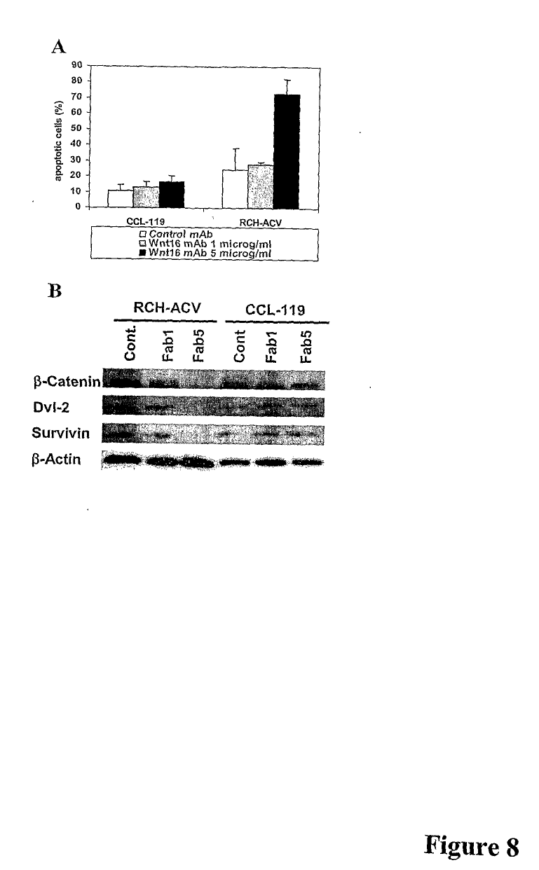

To further confirm and extend the findings above, human anti-Wnt16b antibodies were custom-made. A truncated Wn16 protein (amino acid residues 1-99 of SEQ ID NO:2) was used as an antigen to generate several human Fabs as fully described herein. The Fab antibody's specificity was assessed by showing a high affinity for Wnt16 in both RCH-ACV (FIG. 7) and 697 cells (data not shown). 697 cells, RCH-ACV cells and CCL-119 control cells were treated with the anti-Wnt16 Fab antibody at concentrations of 1 μg/ml and 5 μg/1 ml, respectively and with a control mAb. Apoptotic assay was performed 3-5 days after. 4 independent experiments were performed. Wnt16 Fab induces apoptosis in the same range as Wnt16 siRNA in t(1;19)-containing cells, whereas no effect was noticed in control cell lines (FIG. 8). Moreover, it was confirmed by Western Blot analysis that Wnt16 inhibition induces a decrease in β-catenin, Dvl-2 and survivin expression (FIG. 8B).

It was found that both the mouse antiWnt16 monoclonal antibody and the human anti-Wnt16 Fab antibody could cause significant apoptotic cell death in RCH-ACV cells, while they caused minimal effect on the control leukemia cell lines NALM-6 and CCL-119 (FIGS. 5, 8).

### Example 9

### Anti-Wnt16b Antibody-Induced Apoptotic Effect is Associated with Status of the Wnt16b Proteins

High level of Wnt16b expression was observed in the leukemia cell lines that were sensitive to anti-Wnt16b antibody treatments. However, in the control leukemia cell line CCL-119 that was not sensitive to the antibody treatment only minimal Wnt16b expression was detected. Moreover, Wnt16b was overexpressed in several cancer cell lines including lung cancer, mesothelioma, melanoma, NSCLC, colon cancer, brain cancer, breast cancer, ovarian cancer, cervical cancer, leukemia, lymphoma and non-small-cell lung cancer tissues.

### Example 10

### Anti-Wnt16b Antibody-Induced Apoptosis is Associated with Down-Regulation of Dvl-3, Cytosolic β-catenin, and Survivin

Wnt signaling has been shown to activate β-catenin/Tcf-mediated transcription through Dvl. Wnt signaling also stabilizes cytosolic β-catenin. Thus, it was determined whether anti-Wnt16b antibody-induced apoptosis was dependent on Dvl and destabilization of cytosolic β-catenin. It was found that both Dvl and cytosolic β-catenin level was dramatically down regulated after anti-Wnt16b antibody treatment in the cancer cells that were examined, such as the RCH-ACV cell line. In contrast, no change of both Dvl and cytosolic β-catenin level was found in the control cell line after anti-Wnt16b antibody treatment.

### Example 11

### Wnt16 siRNA-Induced Apoptosis is Associated with Releasing of Smac/Diablo and Cytochrome c from Mitochondria to the cCtosol and JNK Activation

To further elucidate the mechanism through which anti-Wnt16 antibody induces apoptosis in human cancer cells, additional components in the apoptotic pathway were examined. During apoptosis, Smac/Diablo (second mitochondria-derived activator of caspase/direct IAP-binding protein with low pI) functions to remove the IAP-mediated caspase inhibition (Du et al., Cell 102(1):33-42 (2000); Verhagen et al., Cell 102(1):43-53 (2000)). Stimulation of apoptosis causes releasing of Smac/Diablo from the intermembrane space of mitochondria into the cytosol, together with cytochrome c. Cytochrome c directly activates Apaf-1 and caspase-9 and Smac/Diablo interacts with multiple IAPs to remove IAP-mediated inhibition of both initiator and effector caspases (Chai et al., Nature 406(6798):855-62 (2000); Srinivasula et al., J Biol Chem 275(46):36152-7 (2000)).

Consistent with the results above, where caspase-3 activity increases in the cancer cells, but not in the normal cells, an increased level of both Smac/Diablo and cytochrome c was found in the cytosol of the cancer cells after anti-Wnt16 antibody treatment, but not in that of the normal cells. These results indicate that both Smac/Diablo and cytochrome c are likely to be involved in the anti-Wnt16 antibody induced apoptosis by removing survivin and/or other IAPs-mediated inhibition and direct activation of caspases, respectively.

### Discussion

As noted above, little is known regarding the role that Wnt ligand plays in human carcinogenesis. The data presented here demonstrate that Wnt16 signaling play a causal role in human cancer cells and thus, Wnt16 represents a therapeutic target for the treatment of cancer.

The data presented above demonstrate that the Wnt16b siRNA induces apoptosis in human cancer cells. Furthermore, our data indicates that the anti-tumor effect was due to the blockade of Wnt signaling pathway. The apoptotic cell death induced by Wnt16b siRNA was not only correlated with the Wnt16b mRNA expression, but also consistent with the decreased Dvl and cytosolic β-catenin protein expression in the human pre-B ALL cells.

The t(1;19) chromosomal translocation results in the production of a fusion production E2A-Pbx1. E2A-Pbx1 contains the strong transactivation domains of E2a and the DNA binding homeodomain of Pbx1. The activated oncoprotein E2a-Pbx1 induces transformation in several cell types in vitro and induces lymphoblastic lymphomas in transgenic mice (Kamps et al., Genes Dev 5:358-368 (1991)). Recently, expression profiles obtained using microarrays identified t(1;19) (E2A-Pbx1) as one of the 6 prognostic subtypes of pediatric ALL (Yeoh et al., Cancer Cell 1:133-143 (2002)). E2A-Pbx1 is known to induce tumor formation in nude mice Kamps et al. (supra) and to promote T cell lymphomas and myeloid leukemias in thymocytes and myeloid cells, respectively (Kamps, et al., Oncogene 12:19-30 (1996)). However, the mechanisms by which E2A-Pbx1 produces pre-B cell ALL still remain unclear (Aspland et al., Oncogene 20:5708-5717 (2001)).

Wnt16b is of paramount importance in this subtype of leukemia. A role for Wnt signaling has already been demonstrated in hematopoiesis. First, it has been reported that some Wnt proteins stimulate the proliferation of hematopoietic progenitors (Austin et al., Blood 89:3624-3635 (1997); Van Den Berg et al., Blood 92:3189-3202 (1998)). Additionally, Reya et al. showed that Wnt proteins are mitogenic for pro-B cells by activating LEF-1 (Reya et al., Immunity 13:15-24 (2000)). The specific function of Wnt16 was first addressed by McWhirter et al. who proposed that Wnt16 might contribute to the development of t(1;19) pre-B ALL (McWhirte et al., Proc Natl Acad Sci USA 96:11464-11469 (1999)). Using custom diagnostic microarrays, Ross et al. reported significant overexpression of Wnt16 ranging from a 569- to 2547-fold change when comparing the mean signal value in the E2A-Pbx1-expressing leukemia to other leukemia (Ross et al., Blood 102:2951-2959 (2003)). Wnt16 is also overexpressed in chronic lymphocytic leukemia (Lu et al., Proc Natl Acad Sci USA 101:3118-3123 (2004)). Other putative targets for E2a-Pbx1 have been proposed. Fu et al., first analyzed genes induced by E2a-Pbx1 in fibroblasts and identified some tissue-specific and developmentally regulated genes (Fu et al., Mol Cell Biol 17:1503-1512 (1997)). In human t(1;19)-containing pre-B-cell ALL, it was reported that EB-1 overexpression could interfere with proliferation or differentiation (Fu et al., Oncogene 18:4920-4929 (1999)). Recently, Smith et al. demonstrated that E2a-Pbx1 induction enhances expression of Bmi-1, a lymphoid oncogene whose product functions as a transcriptional repressor of the INK4A-ARF tumor suppressor locus (Smith et al., Mol Cell 12:393-400 (2003)). Based on microarray data, Downing also proposed C-Mer as a testable potential target due to its overexpression and its ability to cause transformation (Carroll et al., Hematology (Am Soc Hematol Educ Prgm): 102-131 (2003)). The respective role of these putative target genes and their likely interactions remain largely unknown and warrant further investigations in order to define which one(s) could represent a relevant clinical target.

The data here demonstrate, for the first time, an important function of Wnt16 in leukemogenesis. Fear et al., using in silico bioinformatic gene prediction techniques, identified two Wnt16 isoforms called Wnt16a and Wnt16b (Fear et al., Biochem Biophys Res Comm 278:814-820 (2000)). Both Wnt16 isoforms share 3 of 4 exons, differing only in the composition of their 5′-UTR and first exon. Despite their high homology, both isoforms display very distinct expression patterns in human tissues: Wnt16a is expressed only in the pancreas whereas Wnt16b is expressed more ubiquitously. The specific function of Wnt16a and Wnt16b in oncogenesis was previously unknown. McWhirter et al. demonstrated upregulation of Wnt16 by E2A-Pbx1 in ALL. Interestingly, the Wnt16 gene described in their publication is 100% homologous to Wnt16b. Paradoxically, Fear et al. reported that the Wnt16a promoter contains three consensus binding sites for the oncogenic domain transcription factor E2A-Pbx1. We confirm here by using specific primers for both isoforms and DNA sequencing that Wnt16b and not Wnt16a is over expressed in ALL cell lines and is targeted by E2A-Pbx1. Like other Wnt proteins such as Wnt2, Wnt5 or Wnt7, we show that the two isoforms of the same Wnt16 protein have distinct roles adding to the complexity of the Wnt signaling paradigm (Lustig and Behrens, J Cancer Res Clin Oncol 129:199-221 (2003)).

Through the frizzled receptor and dishevelled protein, Wnt signaling activates two distinct pathways: the canonical pathway (i.e., β-catenin pathway) and the JNK pathway. Dishevelled protein has three highly conserved domains, DIX, PDZ, and DEP. Among them, the DIX and PDZ domains are necessary for the canonical signaling pathway while the DEP domain is important for the activation of JNK pathway.

It has been suggested that the activation of JNK plays a critical role in initiating apoptosis (Wang et al., Mol Cell Biol 19(9):5923-9 (1999)). Recently, Chen et al. have demonstrated that Wnt1 inhibits apoptosis by activating β-catenin and TCF transcription (Chen et al., J Cell Biol 152(1):87-96 (2001)). In this study, both overexpression of β-catenin and increased JNK activity were observed after anti-Wnt16 antibody treatment, suggesting that both the canonical pathway and the JNK pathway are involved in the apoptosis induced by anti-Wnt16 antibody. In addition, overexpression of Dvl in a normal mesothelial cell line down regulated JNK activities and the inhibition of Dvl by using Apigenin to block CK-2 activity increased JNK activity. Most likely, the activation of JNK after anti-Wnt16 antibody treatment is through Dvl.

We here used two different techniques to inhibit Wnt16: silencing using siRNA and antibody inhibition. Both approaches lead to the same observations: inhibition of Wnt16 induces apoptosis through the canonical Wnt pathway. This indicates that aberrant Wnt signaling mediated by Wnt16 contributes to the failing in apoptosis that is the signature of this highly aggressive malignancy. The above findings raise the importance of Wnt signaling in oncogenesis and extend our previous findings in solid tumors to hematopoietic malignancies. New insights into the role of Wnt16 in this subset of leukemia displaying the t(1;19) translocation is provided. The findings presented herein and especially the potency of anti-Wnt16 antibody to inhibit Wnt16 expression support the therapeutic interest of targeting Wnt16 in this disease.

The above findings suggest that Wnt16 antibodies may not only induce directly apoptosis in cancer cell that over express Wnt16 proteins, but also release potential drug resistance by restoring normal apoptotic machinery back to these tumor cells. The basis for drug resistance in tumor cells is most likely the disruption of apoptosis. Overexpression of Survivin, an inhibitor of apoptosis, is a common feature of most human cancers. It has been shown that targeting of survivin increases the sensitivity of tumor cells to cytotoxic drugs (Grossman et al., Proc Natl Acad Sci USA 98(2):635-40 (2001)). Further, it has been shown that antisense survivin is sufficient to cause apoptosis in human mesothelioma cells. Moreover, a synergistic effect between antisense survivin and chemotherapy has also been reported.

Here, it is shown that anti-Wnt16 antibody treatment dramatically decreases the protein expression level of Survivin. Taken together, Wnt16 antibody could potentiate and synergize the effect of standard chemotherapy in human cancer cells.

Other antagonists of Wnt signaling or Frizzled receptor should also induce apoptosis through dishevelled. For instance, sFRPs function as soluble modulators of Wnt signaling by competing with the Frizzled receptors for the binding of secreted Wnt ligands (Melkonyan et al., Proc Natl Acad Sci USA 94(25):13636-41 (1997)). Specifically, sFRPs can either antagonize Wnt function by binding the protein and blocking access to its cell surface signaling receptor, or they can enhance Wnt activity by facilitating the presentation of ligand to the Frizzled receptors (Uthoff et al., Mol Carcinog 31 (1):56-62 (2001)). Frizzled receptor antagonists (e.g., antibody specific for the extracellular domain or small molecule specific for the intracellular domain) should induce apoptosis in human cancer cells that over express Wnt/frizzled proteins.

### Example 12

### Anti-Wnt16 Antibodies Induce Apoptosis in Human Lung Cancer Cells

This example shows that Wnt16 antibodies of the invention induce apoptosis in human lung cancer cells. In addition, western blot analysis was used to demonstrate expression of Wnt16 in other cancer cells. These experiments were carried out generally using the materials and methods described in Example 1.

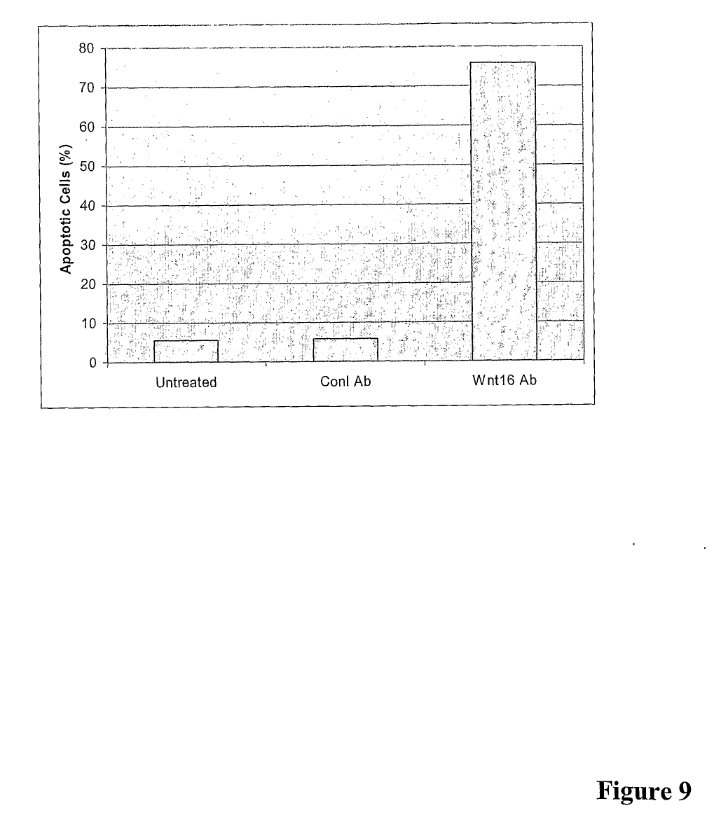

FIG. 9 shows induction of apoptotic cell death after treatment with a mouse anti-Wnt16 monoclonal antibody in human lung cancer cell line H460 (lung adenocarcinoma). H460 cells were treated for three days with no antibody (untreated), control IgG antibody (Conl Ab; 5 μg/mL; MOPs, Sigma Chemicals), or anti-Wnt16 monoclonal antibody (Wnt16 Ab; 5 μg/mL; BD Pharmingen, cat #552595). Apoptosis was analyzed using the flow cytometry. Treatment with the mouse anti-Wnt16 monoclonal antibody induced apoptosis in 75.9% cells, as compared with 5.59% and 5.86% observed from cells untreated or treated with a control IgG antibody, respectively. In a separate experiment, H460 cells were incubated with 10 μg/ml of the anti-Wnt16 monoclonal antibody and the following result was obtained: H460 cells incubated without antibody treatment showed 5.7% apoptotic cells; H460 cells incubated with control IgG showed 6% apoptotic cells; and cells incubated with the mouse anti-Wnt16 monoclonal antibody showed 97.5% apoptotic cells.

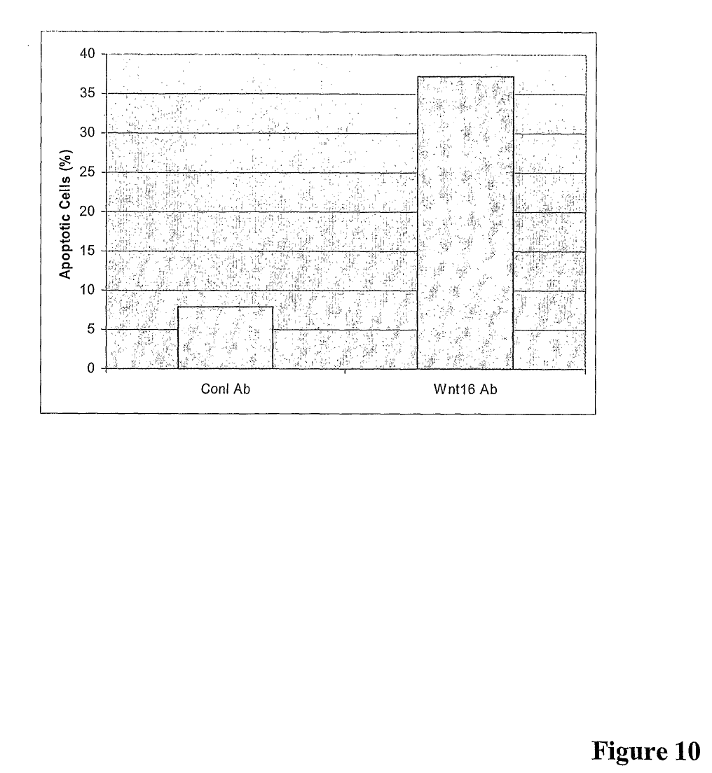

In a similar experiment as described above, H460 cells were incubated with a human anti-Wnt16 antibody, Fab clone #585 (see above), FIG. 10 shows induction of apoptotic cell death after treatment with the human anti-Wnt16 Fab clone #585 in human lung cancer cell line H460. H460 cells were treated for three days with control IgG antibody (Conl Ab; 5 ug/mL), or the human Wnt16 antibody (Wnt16 Ab; 5 ug/mL; clone #585). Apoptosis was analyzed using flow cytometry. Treatment with Wnt16 antibody Fab clone #585 induced apoptosis in 37.3% cells, as compared with 7.92% in cells treated with controlled IgG antibody.

In summary, these results show that anti-Wnt16 monoclonal antibodies induce tumor-specific apoptosis in human cancer cells, probably through both the canonical and the JNK pathways.

### Example 13

### Wnt16 Protein is Expressed in Cancer Cells

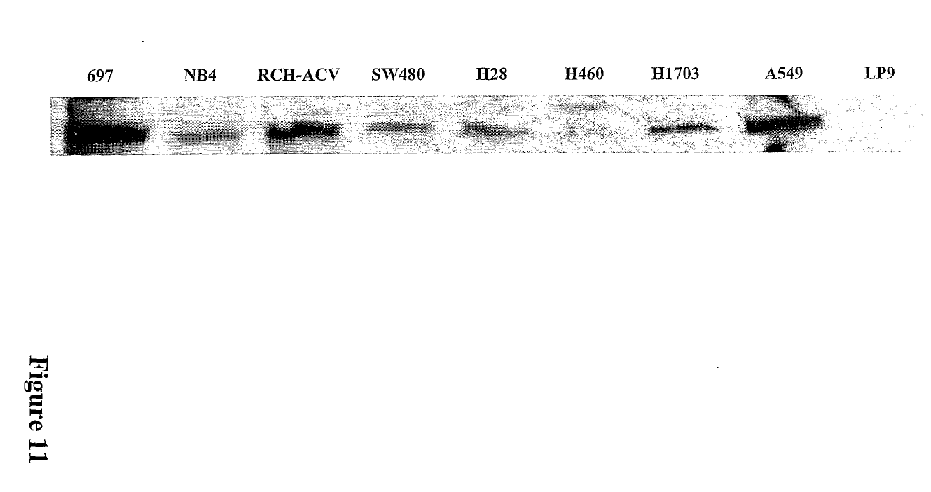

In order to analyze Wnt16 expression in various cancer cells, Western Blotting using a Wnt16 polyclonal antibody was performed. Cancer cell lines tested include leukemia cell lines 697, NB4, and RCH-ACV; colon cancer cell line SW480; mesothelioma cell line H28; lung cancer cell lines H460, H1703, and A549; and a normal mesothelial cell line, LP9. FIG. 11 shows that Wnt16 protein is expressed in various human cancer cell lines, while no Wnt16 expression was detected in LP9 cells.

### Example 14

### Wnt16 mRNA is Expressed in Cancer Cells

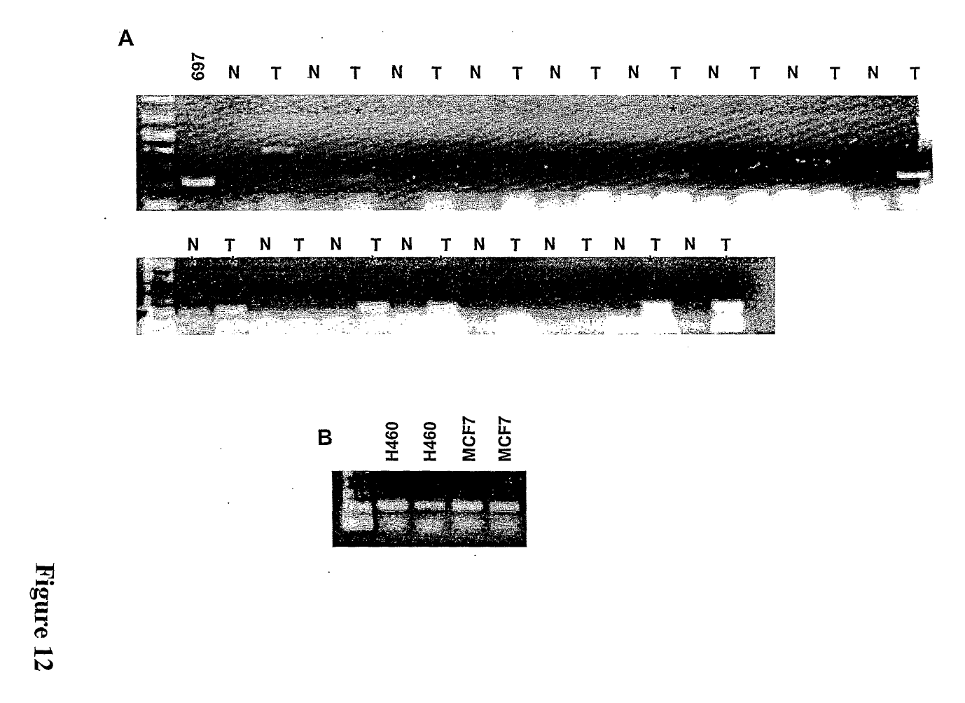

Wnt16 mRNA expression was analyzed in non-small cell lung cancer and cell lines using RT-PCR. Total RNA was isolated from primary lung cancer and normal tissues. RT-PCR was carried out for 30-35 cycles and analyzed by gel electrophoresis. Upon analyzing 17 paired RNA samples, 8 out of 17 tumor samples and one normal sample showed the expected PCR fragment (FIG. 12A). Wnt16 mRNA expression was also shown in the breast cancer cell line MCF7 (FIG. 12B).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of inhibiting the proliferation of a cell that overexpresses a Wnt16, the method comprising the step of contacting the cell with an amount of an agent that inhibits Wnt16 signaling effective to inhibit proliferation of the cell.

2. The method of claim 1, wherein the cell is a cancer cell.

3. The method of claim 2, wherein the cancer cell is selected from the group consisting of lung, mesothelioma, melanoma, colon, brain, breast, ovarian, cervical, leukemia, lymphoma and non-small-cell lung cancer cells.

4. The method of claim 3, wherein the cancer cell is a leukemia cell.

5. The method of claim 4, wherein the cancer cell comprises a t(1;19) translocation.

6. The method of claim 4, wherein the leukemia cell is an acute lymphoblastoid leukemia cell, a pre-B-cell acute lymphoblastoid leukemia cell or a B cell chronic lymphocytic leukemia cell.

7. The method of claim 3, wherein the cancer cell is a lung cancer cell.

8. The method of claim 1, wherein the agent is a siRNA.

9. The method of claim 1, wherein the agent is an anti-Wnt16 antibody.

10. The method of claim 9, wherein the antibody specifically binds to a Wnt16 protein.

11. The method of claim 10, wherein the Wnt16 protein is a human Wnt16b protein.

12. The method according to claim 9, wherein the anti-Wnt16 antibody binds a polypeptide consisting of amino acid sequence corresponding to amino acid residues 1-99 of human Wnt16 as shown in SEQ ID NO:2.

13. The method according to claim 9, wherein the anti-Wnt16 antibody specifically binds a polypeptide consisting of amino acid residues 1-99 of human Wnt16 as shown in SEQ ID NO:2.

14. The method of claim 9, wherein the anti-Wnt16 antibody binds a polypeptide comprising an amino acid sequence as shown in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16.

15. The method of claim 9, wherein the anti-Wnt16 antibody competes for binding a Wnt16 with a second anti-Wnt16 antibody that specifically binds a polypeptide consisting of amino acid residues 1-99 of human Wnt16 as shown in SEQ ID NO:2.

16. The method of claim 9, wherein the anti-Wnt16 antibody competes for binding a Wnt16 with a second anti-Wnt16 antibody that specifically binds a polypeptide comprising an amino acid sequence as shown in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16.

17. The method of claim 9, wherein the anti-Wnt16 antibody is a polyclonal antibody.

18. The method of claim 9, wherein the anti-Wnt16 antibody is a monoclonal antibody.

19. The method of claim 18, wherein the anti-Wnt16 antibody is a mouse monoclonal antibody.

20. The method of claim 9, wherein the anti-Wnt16 antibody is a chimeric antibody.

21. The method of claim 9, wherein the anti-Wnt16 antibody is a humanized antibody.

22. The method of claim 9, wherein the anti-Wnt16 antibody is a human Fab.

23. The method of claim 9, wherein the anti-Wnt16 antibody is a fully human antibody.

24. The method of claim 9, wherein the anti-Wnt16 antibody is recombinantly produced.

25. The method of claim 1 which is practiced in vitro.

26. The method of claim 1 which is practiced in vivo.

27. The method of claim 1, wherein the cell is in a patient and the step of contacting is carried out by administering the agent to the patient.

28. The method of claim 27, wherein the agent is an anti-Wnt16 antibody.

29. The method of claim 27, wherein the agent is a siRNA.

30. The method of claim 27, further comprising administering to the patient a second therapeutic agent.

31. The method of claim 30, wherein the second therapeutic agent is a chemotherapeutic agent.

32. The method of claim 30, wherein the second therapeutic agent is radiation therapy.

33. A method of inducing apoptosis of a cell that overexpresses a Wnt16, comprising the step of contacting the cell with an amount of an agent that inhibits Wnt16 signaling effective to induce apoptosis of the cell.

34. A method of treating a disease associated with Wnt16 signaling comprising administering to a subject in need of such treatment an amount of an agent that inhibits Wnt16 signaling effective to treat the disease.

35. A method of detecting in a biological sample from a patient a cell that overexpresses a Wnt16, the method comprising the step of detecting the level of Wnt16 expression in the biological sample.

36. The method of claim 35, wherein the biological sample is a serum sample.

37. The method of claim 35, wherein the biological sample is a blood, sputum, urine or stool sample.

38. The method of claim 35, wherein the step of detecting the level of Wnt16 expression is carried out by detecting the level of a Wnt16 mRNA.

39. The method of claim 35, wherein the step of detecting the level of Wnt16 expression is carried out by detecting the level of a Wnt16 protein.

40. The method of claim 35, wherein the detection of the level of Wnt16 expression is used to predict response to a therapeutic regimen.

41. The method of claim 40, wherein the therapeutic regimen comprises administering to a patient a monoclonal anti-Wnt16 antibody.

42. A pharmaceutical composition comprising an anti-Wnt16 antibody and a pharmaceutically acceptable excipient, carrier and/or diluent.

43. The pharmaceutical composition of claim 42, wherein the anti-Wnt16 antibody is a polyclonal antibody.

44. The pharmaceutical composition of claim 42, wherein the anti-Wnt16 antibody is a monoclonal antibody.

45. The pharmaceutical composition of claim 42, wherein the anti-Wnt16 antibody is further conjugated to an effector component.

46. The pharmaceutical composition of claim 45, wherein the effector component is a fluorescent label.

47. The pharmaceutical composition of claim 45, wherein the effector component is a radioisotope or a cytotoxic chemical.
